SEQUENCE LISTING (2 sequences)

SEQ ID NO: 1 - protein, 774 aa, unidentified
MAHEAMEYDVQVQLNHAEQQPAPAGMASSQGGPALLQPVPADVVSSQGVPSILQPAPAEVISSQATPPLLQPAPQLSVDLTEVEVLGEDTVENINPRTSEQHRQGSDGNHTIPASSLHSMTNFISGLQRLHGMLEFLRPSSSNHSVGPMRTRRRVSASRRARAGGSQRTDSARLRAPLDAYFQVSRTQPDLPATTYDSETRNPVSEELQVSSSSDSDSDSSAEYGGVVDQAEESGAVILEEQLAGVSAEQEVTCIDGGKTLPKQPSPQKSEPLLPSASMDEEEGDTCTICLEQWTNAGDHRLSALRCGHLFGYRCISTWLKGQVRKCPQCNKKARHSDIVVLYARTLRALDTSEQERMKSSLLKEQMLRKQAELESAQCRLQLQVLTDKCTRLQRRVQDLQKLTSHQSQNLQQPRGSQAWVLSCSPSSQGQHKHKYHFQKTFTVSQAGNCRIMAYCDALSCLVISQPSPQASFLPGFGVKMLSTANMKSSQYIPMHGKQIRGLAFSSYLRGLLLSASLDNTIKLTSLETNTVVQTYNAGRPVWSCCWCLDEANYIYAGLANGSILVYDVRNTSSHVQELVAQKARCPLVSLSYMPRAASAAFPYGGVLAGTLEDASFWEQKMDFSHWPHVLPLEPGGCIDFQTENSSRHCLVTYRPDKNHTTIRSVLMEMSYRLDDTGNPICSCQPVHTFFGGPTCKLLTKNAIFQSPENDGNILVCTGDEAANSALLWDAASGSLLQDLQTDQPVLDICPFEVNRNSYLATLTEKMVHIYKWE

SEQ ID NO: 2 - protein, 560 aa, unidentified
MNRKWEAKLKQIEERASHYERKPLSSVYRPRLSKPEEPPSIWRLFHRQAQAFNFVKSCKEDVHVFALECKVGDGQRIYLVTTYAEFWFYYKSRKNLLHCYEVIPENAVCKLYFDLEFNKPANPGADGKKMVALLIEYVCKALQELYGVNCSAEDVLNLDSSTDEKFSRHLIFQLHDVAFKDNIHVGNFLRKILQPALDLLGSEDDDSAPETTGHGFPHFSEAPARQGFSFNKMFTEKATEESWTSNSKKLERLGSAEQSSPDLSFLVVKNNMGEKHLFVDLGVYTRNRNFRLYKSSKIGKRVALEVTEDNKFFPIQSKDVSDEYQYFLSSLVSNVRFSDTLRILTCEPSQNKQKGVGYFNSIGTSVETIEGFQCSPYPEVDHFVLSLVNKDGIKGGIRRWNYFFPEELLVYDICKYRWCENIGRAHKSNNIMILVDLKNEVWYQKCHDPVCKAENFKSDCFPLPAEVCLLFLFKEEEEFTTDEADETRSNETQNPHKPSPSRLSTGASADAVWDNGIDDAYFLEATEDAELAEAAENSLLSYNSEVDEIPDELIIEVLQE

(19) United States
(12) Patent Application Publication
(10) Pub. No.: US 2026/0159841 A1
(43) Pub. Date: Jun. 11, 2026

(54) COMPOSITIONS AND METHODS FOR TREATMENT OF CANCERS

(71) Applicant: New York University, New York, NY (US)

(72) Inventors: John T. POIRIER, Brooklyn, NY (US); Tony T. HUANG, Long Island City, NY (US); Kwok-Kin WONG, Arlington, MA (US)

(73) Assignee: New York University, New York, NY (US)

(21) Appl. No.: 19/414,970

(22) Filed: Dec. 10, 2025

Related U.S. Application Data
(60) Provisional application No. 63/730,106, filed on Dec. 10, 2024.

(51) Int. Cl. C12N 15/113; A61K 31/7088; A61K 38/46; A61K 45/06; A61P 35/00

(52) U.S. Cl. CPC C12N 15/1137; A61K 31/7088; A61K 38/465; A61K 45/06; A61P 35/00; C12N 2310/11; C12N 2310/141

(57) ABSTRACT

The present disclosure provides methods for treating cancer or for increasing a sensitivity of a plurality of cancer cells to a chemotherapeutic agent.

### CROSS-REFERENCE TO RELATED APPLICATIONS

This patent application claims priority to U.S. Provisional Application No. 63/730,106, filed on Dec. 10, 2024, the disclosure of which is herein incorporated by reference in its entirety.

### STATEMENT REGARDING FEDERALLY SPONSORED RESEARCH

This invention was made with government support under GM139610 and ES031658 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Dec. 5, 2025, is named 243735_000459_SL.xml and is 3,880 bytes in size.

### BACKGROUND

Cancer is a leading cause of morbidity and mortality in the world. While intensive screening efforts have made a measurable impact on cancer early detection, regional and distant metastases at diagnosis remain commonplace. Systemic therapies can treat patients who are not candidates for local control with surgery or radiation therapy, or to downstage locally advanced tumors, aiming to render them operable. Among the subset of patients who are candidates for local control, adjuvant systemic therapy significantly reduces disease recurrence.

In recent years small molecule inhibitors with selectivity for mutant oncogenes, antibody-based therapies, and immune checkpoint blockade have been approved clinically. For many patients, this change in clinical practice has altered the course of their disease; however, one study estimates that more than 85% of cancer patients are not candidates for precision medicine. A cross-sectional study of the nearly half of U.S. patients eligible for immune checkpoint blockade concluded that about 87% of treatment eligible patients did not respond. Chemotherapy is therefore likely to remain a significant component of cancer treatment alone and in combination with other treatment modalities.

### SUMMARY

Recognized herein is the issue that durable responses to chemotherapy are infrequent or otherwise suboptimal in some cancers. This variability in outcomes is not fully explained by somatic defects in DNA damage response and repair genes alone, and is instead likely driven by a combination of genetic and epigenetic factors. Recognized herein is the finding that increased understanding of the underlying mechanistic basis of chemosensitivity and chemoresistance may help improve treatment outcomes.

An aspect of the present disclosure provides a method for treating cancer in a subject, the method comprising: (a) decreasing one or both of (i) an amount of an endogenous ubiquitin ligase in cancerous cells of a subject and (ii) ligase activity of the endogenous ubiquitin ligase in cancerous cells of the subject, wherein the endogenous ubiquitin ligase has at least 80% sequence identity to SEQ ID NO: 1; and (b) administering a therapeutically effective amount of a chemotherapeutic agent to the subject. In some embodiments, the subject has, prior to (a), been identified as having reduced expression or activity of Schlafen 11. In some embodiments, reduced expression or activity of Schlafen 11 is determined by comparing a level of Schlafen 11 expression to a threshold value. In some embodiments, the cancerous cells exhibit less than 1% Schlafen 11 positivity by immunochemistry. In some embodiments, reduced expression or activity of Schlafen 11 is determined by comparing a level of Schlafen 11 activity to a threshold value. In some embodiments, the method comprises decreasing an amount of the endogenous ubiquitin ligase in cancerous cells of the subject. In some embodiments, decreasing the amount of the endogenous ubiquitin ligase comprises knocking out a gene that encodes for the endogenous ubiquitin ligase. In some embodiments, the gene is knocked out via CRISPR, TALEN, or zinc-finger nuclease DNA editing. In some embodiments, decreasing the amount of the endogenous ubiquitin ligase comprises post-transcriptional gene silencing (e.g., RNAi). In some embodiments, decreasing the amount of the endogenous ubiquitin ligase comprises delivering a microRNA (miRNA) or a precursor thereof to the subject, wherein the miRNA is configured to bind to an mRNA encoding the endogenous ubiquitin ligase. In some embodiments, decreasing the amount of the endogenous ubiquitin ligase comprises delivering an antisense oligonucleotide or a precursor thereof to the subject, wherein the antisense oligonucleotide is configured to bind to an mRNA encoding the endogenous ubiquitin ligase. In some embodiments, the method comprises decreasing ligase activity of the endogenous ubiquitin ligase in cancerous cells of the subject. In some embodiments, decreasing the activity of the endogenous ubiquitin ligase comprises administering an inhibitor of the endogenous ubiquitin ligase to the subject. In some embodiments, the chemotherapeutic agent is a drug that induces replication stress during S phase. In some embodiments, the chemotherapeutic agent is selected from the group consisting of a PARP inhibitor, a DNA alkylating agent, a DNA crosslinker, a topoisomerase I inhibitor, a topoisomerase II inhibitor, a polymerase inhibitor, a polymerase alpha inhibitor, and a clastogen. In some embodiments, the cancerous cells exhibit unrestrained fork restart. In some embodiments, the cancerous cells exhibit single-strand DNA replication gaps after fork restart. In some embodiments, the cancerous cells are small cell lung cancer cells. In some embodiments, the cancerous cells are from a neuroendocrine tumor. In some embodiments, the neuroendocrine tumor is a high-grade neuroendocrine tumor. In some embodiments, the subject had been previously treated with a chemotherapeutic agent prior to (a), and the cancerous cells exhibited chemoresistance prior to (a). In some embodiments, the endogenous ubiquitin ligase has at least 85%, at least 90%, at least 95%, at least 96%, at least 97%, at least 98%, at least 99%, or 100% sequence identity to SEQ ID NO: 1.

Another aspect of the present disclosure provides a method for sensitizing a plurality of cancerous cells to a chemotherapeutic agent, the method comprising: (a) decreasing one of both of (i) an amount of an endogenous ubiquitin ligase in a plurality of cancerous cells or (ii) ligase activity of the endogenous ubiquitin ligase in the cancerous cells, wherein the endogenous ubiquitin ligase has at least 80% sequence identity to SEQ ID NO: 1; and (b) contacting the cells with a chemotherapeutic agent. In some embodiments, the cancerous cells have, prior to (a), been identified as having reduced expression or activity of Schlafen 11. In some embodiments, reduced expression or activity of Schlafen 11 is determined by comparing a level of Schlafen 11 expression to a threshold value. In some embodiments, the cancerous cells exhibit less than 1% Schlafen 11 positivity by immunochemistry. In some embodiments, reduced expression or activity of Schlafen 11 is determined by comparing a level of Schlafen 11 activity to a threshold value. In some embodiments, the method comprises decreasing an amount of the endogenous ubiquitin ligase in the cancerous cells. In some embodiments, decreasing the amount of the endogenous ubiquitin ligase comprises knocking out a gene that encodes for the endogenous ubiquitin ligase. In some embodiments, the gene is knocked out via CRISPR, TALEN, or zinc-finger nuclease DNA editing. In some embodiments, decreasing the amount of the endogenous ubiquitin ligase comprises post-transcriptional gene silencing (e.g., RNAi). In some embodiments, decreasing the amount of the endogenous ubiquitin ligase comprises delivering a microRNA (miRNA) or a precursor thereof to the cancerous cells, wherein the miRNA is configured to bind to an mRNA encoding the endogenous ubiquitin ligase. In some embodiments, decreasing the amount of the endogenous ubiquitin ligase comprises delivering an antisense oligonucleotide or a precursor thereof to the cancerous cells, wherein the antisense oligonucleotide is configured to bind to an mRNA encoding the endogenous ubiquitin ligase. In some embodiments, the method comprises decreasing ligase activity of the endogenous ubiquitin ligase in the cancerous cells. In some embodiments, decreasing the activity of the endogenous ubiquitin ligase comprises administering an inhibitor of the endogenous ubiquitin ligase to the cancerous cells. In some embodiments, the chemotherapeutic agent is a drug that induces replication stress during S phase. In some embodiments, the chemotherapeutic agent is selected from the group consisting of a PARP inhibitor, a DNA alkylating agent, a DNA crosslinker, a topoisomerase I inhibitor, a topoisomerase II inhibitor, a polymerase inhibitor, a polymerase alpha inhibitor, and a clastogen. In some embodiments, the cancerous cells exhibit unrestrained fork restart. In some embodiments, the cancerous cells exhibit single-strand DNA replication gaps after fork restart. In some embodiments, the cancerous cells are small cell lung cancer cells. In some embodiments, the cancerous cells are from a neuroendocrine tumor. In some embodiments, the neuroendocrine tumor is a high-grade neuroendocrine tumor. In some embodiments, the cancerous cells had been previously treated with a chemotherapeutic agent prior to (a), and the cancerous cells exhibited chemoresistance prior to (a). In some embodiments, the endogenous ubiquitin ligase has at least 85%, at least 90%, at least 95%, at least 96%, at least 97%, at least 98%, at least 99%, or 100% sequence identity to SEQ ID NO: 1.

Another aspect of the present disclosure provides a method for treating cancer in a subject, the method comprising: (a) decreasing one or both of (i) an amount of an endogenous enzyme having DNA polymerase and DNA primase activity (PrimPol) in cancerous cells of a subject and (ii) primase and/or polymerase activity of the endogenous enzyme having DNA polymerase and DNA primase activity in cancerous cells of the subject, wherein the enzyme has at least 80% sequence identity to SEQ ID NO: 2; and (b) administering a therapeutically effective amount of a chemotherapeutic agent to the subject. In some embodiments, the subject has, prior to (a), been identified as having reduced expression or activity of Schlafen 11. In some embodiments, reduced expression or activity of Schlafen 11 is determined by comparing a level of Schlafen 11 expression to a threshold value. In some embodiments, the cancerous cells exhibit less than 1% Schlafen 11 positivity by immunochemistry. In some embodiments, reduced expression or activity of Schlafen 11 is determined by comparing a level of Schlafen 11 activity to a threshold value. In some embodiments, the method comprises decreasing an amount of the enzyme in cancerous cells of the subject. In some embodiments, decreasing the amount of the enzyme comprises knocking out a gene that encodes for the enzyme. In some embodiments, the gene is knocked out via CRISPR, TALEN, or zinc-finger nuclease DNA editing. In some embodiments, decreasing the amount of the enzyme comprises post-transcriptional gene silencing (e.g., RNAi). In some embodiments, decreasing the amount of the enzyme comprises delivering a microRNA (miRNA) or a precursor thereof to the subject, wherein the miRNA is configured to bind to an mRNA encoding the enzyme. In some embodiments, decreasing the amount of the enzyme comprises delivering an antisense oligonucleotide or a precursor thereof to the subject, wherein the antisense oligonucleotide is configured to bind to an mRNA encoding the enzyme. In some embodiments, the method comprises decreasing primase activity of an enzyme in cancerous cells of the subject. In some embodiments, the method comprises decreasing polymerase activity of an enzyme in cancerous cells of the subject. In some embodiments, decreasing the activity of the enzyme comprises administering an inhibitor of the enzyme to the subject. In some embodiments, the chemotherapeutic agent is a drug that induces replication stress during S phase. In some embodiments, the chemotherapeutic agent is selected from the group consisting of a PARP inhibitor, a DNA alkylating agent, a DNA crosslinker, a topoisomerase I inhibitor, a topoisomerase II inhibitor, a polymerase inhibitor, a polymerase alpha inhibitor, and a clastogen. In some embodiments, the cancerous cells exhibit unrestrained fork restart. In some embodiments, the cancerous cells exhibit single-strand DNA replication gaps after fork restart. In some embodiments, the cancerous cells are small cell lung cancer cells. In some embodiments, the cancerous cells are from a neuroendocrine tumor. In some embodiments, the neuroendocrine tumor is a high-grade neuroendocrine tumor. In some embodiments, the subject had been previously treated with a chemotherapeutic agent prior to (a), and the cancerous cells exhibited chemoresistance prior to (a). In some embodiments, the enzyme has at least 85%, at least 90%, at least 95%, at least 96%, at least 97%, at least 98%, at least 99%, or 100% sequence identity to SEQ ID NO: 2.

Another aspect of the present disclosure provides a method for sensitizing a plurality of cancerous cells to a chemotherapeutic agent, the method comprising: (a) decreasing one of both of (i) an amount of an endogenous enzyme having DNA polymerase and DNA primase activity (PrimPol) in a plurality of cancerous cells or (ii) primase and/or polymerase activity of the endogenous enzyme having DNA polymerase and/or DNA primase activity in the cancerous cells, wherein the enzyme has at least 80% sequence identity to SEQ ID NO: 2; and (b) contacting the cells with a chemotherapeutic agent. In some embodiments, the cancerous cells have, prior to (a), been identified as having reduced expression or activity of Schlafen 11. In some embodiments, reduced expression or activity of Schlafen 11 is determined by comparing a level of Schlafen 11 expression to a threshold value. In some embodiments, the cancerous cells exhibit less than 1% Schlafen 11 positivity by immunochemistry. In some embodiments, reduced expression or activity of Schlafen 11 is determined by comparing a level of Schlafen 11 activity to a threshold value. In some embodiments, the method comprises decreasing an amount of an enzyme in the cancerous cells. In some embodiments, decreasing the amount of the enzyme knocking out a gene that encodes for the enzyme. In some embodiments, the gene is knocked out via CRISPR, TALEN, or zinc-finger nuclease DNA editing. In some embodiments, decreasing the amount of the enzyme comprises post-transcriptional gene silencing (e.g., RNAi). In some embodiments, decreasing the amount of the enzyme comprises delivering a microRNA (miRNA) or a precursor thereof to the cancerous cells, wherein the miRNA is configured to bind to an mRNA encoding the enzyme. In some embodiments, decreasing the amount of the enzyme comprises delivering an antisense oligonucleotide or a precursor thereof to the cancerous cells, wherein the antisense oligonucleotide is configured to bind to an mRNA encoding the enzyme. In some embodiments, the method comprises decreasing DNA primase activity of an enzyme in the cancerous cells. In some embodiments, the method comprises decreasing DNA polymerase activity of an enzyme in the cancerous cells. In some embodiments, decreasing the activity of the enzyme comprises administering an inhibitor of the enzyme to the cancerous cells. In some embodiments, the chemotherapeutic agent is a drug that induces replication stress during S phase. In some embodiments, the chemotherapeutic agent is selected from the group consisting of a PARP inhibitor, a DNA alkylating agent, a DNA crosslinker, a topoisomerase I inhibitor, a topoisomerase II inhibitor, a polymerase inhibitor, a polymerase alpha inhibitor, and a clastogen. In some embodiments, the cancerous cells exhibit unrestrained fork restart. In some embodiments, the cancerous cells exhibit single-strand DNA replication gaps after fork restart. In some embodiments, the cancerous cells are small cell lung cancer cells. In some embodiments, the cancerous cells are from a neuroendocrine tumor. In some embodiments, the neuroendocrine tumor is a high-grade neuroendocrine tumor. In some embodiments, the cancerous cells had been previously treated with a chemotherapeutic agent prior to (a), and the cancerous cells exhibited chemoresistance prior to (a). In some embodiments, the enzyme has at least 85%, at least 90%, at least 95%, at least 96%, at least 97%, at least 98%, at least 99%, or 100% sequence identity to SEQ ID NO: 2.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Novel features of the disclosure are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIGS. 1A-C depicts dose-responsive curves for three different cell lines treated with hydroxyurea (HU) (FIG. 1A), camptothecin (FIG. 1B) and mitomycin C (FIG. 1C) in the presence or absence of doxycycline. The three cell lines were HAP1 parental clones, two Schlafen 11 (SLFN11) knockout clones, and two knockout clones reconstituted with SLFN11 cDNA under control of a doxycycline-inducible promoter. Doxycycline induced a ˜5-fold increase in sensitivity to both drugs in the reconstituted SLFN11 knockout cell. Doxycycline caused no further increase in chemosensitivity in any other condition.

FIGS. 2A-C depict the results of a DNA fiber analysis to test fork speed in SLFN11-expressing parental HAP1 cells and three different CRISPR knockout cell lines that were disrupted in exon 1, 3 or 4 of SLFN11.

FIGS. 3A-D depict DNA fiber assay and a standard pulse-chase experiment measuring replication fork restart in SLFN11-expressing parental HAP1 cells and three different CRISPR knockout cell lines that were disrupted in exon 1, 3 or 4 of SLFN11. Specifically, incorporation of a CIdU thymidine analog (CIdU tract length) after replication fork stalling by hydroxyurea treatment (HU) is measured relative to IdU incorporation prior to fork stalling. The results showed that, in contrast to SLFN11 proficiency, loss of SLFN11 in HAP1 cells causes highly efficient recovery of stalled forks using the DNA fiber fork restart assay depicted in FIG. 3A. The fork restart efficiency is measured by depicting the CIdU tract length (recovered forks) between SLFN11 proficient or deficient HAP1 cells (FIG. 3C) or by comparing the ratio of the recovered forks (CIdU tracts) with the IdU tracts (prior to HU treatment) (FIG. 3D). Longer CIdU tract lengths or higher CIdU to IdU ratio depicts more efficient fork recovery from stalled forks.

FIG. 4 depicts immunoblotting results for HAP1 cells, parental HAP1 or SLFN11 KO cells treated with 2 mM HU for 4 hours.

FIG. 5 depicts immunoblotting results for SLFN11/RFWD3 double knockout cell lines.

FIGS. 6A-C depict an analysis of fork speed following restart in SLFN11/RFWD3 double knockout cell lines. Specifically, FIG. 6A depicts a western blot, FIG. 6B depicts the design of a fork restart experiment, and FIG. 6C depicts the fork restart data. SLFN11/RFWD3 double knockout cells (FIG. 6A) phenocopy the SLFN11 parental fork restart phenotype, suggesting that SLFN11 antagonizes RFWD3 activity (FIGS. 6B-C).

FIG. 7 depicts dose-responsive curves of SFLN11/RFWD3 double knockout cell lines treated with hydroxyurea, camptothecin, and mitomycin C. SLFN11/RFWD3 double knockout cells exhibit sensitivity to camptothecin, mitomycin C, and hydroxyurea that is comparable to the parental, SLFN11-expressing cell lines.

FIGS. 8A-C depict a DNA fiber assay for DNA fibers digested with S1 nuclease, which revealed ssDNA replication gaps by collapsing extended CIdU replication tracks. S1 nuclease digestion revealed that SLFN11 CIdU tracks were replete with ssDNA gaps, implicating PrimPol.

FIGS. 9A-E depict the results of analyzing PrimPol for its role in restart of stalled replication forks. Even partial depletion of PrimPol in SLFN11 knockout cell lines phenocopied the parental, SLFN11-expressing cell lines as well as SLFN11/RFWD3 double knockout cell lines with respect to RPA32 ubiquitination (FIG. 9A), fork restart relative to initial fork speed (FIG. 9B), percent of stalled forks (FIG. 9D), and absolute tract length in kilobases (FIG. 9E).

FIGS. 10A-D depict the results of SLFN11 and PrimPol RNAi experiments performed in the small cell lung cancer cell line SW1271. DNA fiber assay was performed to test the role of SLFN11 and Primpol siRNA knockdown for their effects on replication fork restart tract length in SW1271 cells. The results showed that, similar to the SLFN11 knockout HAP1 cell lines, the transient depletion of SLFN11 in SW1271 cells increased the tract length of restarted forks. This phenotype was dependent on Primpol as Primpol and SLFN11 double knockdown reduced the tract length of restarted forks back to control levels (SW1271 siCtrl) (FIG. 10C). Western blot analysis confirmed siRNA knockdown efficiencies (FIG. 10B) in SW1271 cells, and examples of DNA fiber labeling schematics and tract lengths are shown in FIGS. 10A and D, respectively.

FIGS. 11A-D depict the results of siRNA to knock down PrimPol in two cell lines that do not express SLFN11: U2OS and RPE cells. FIG. 11A depicts Western blot results and FIGS. 11B-D depict the design and results of DNA fiber analysis to measure fork restart. These results phenocopied HAP1 parental cell lines, which express SLFN11, and recapitulated the results observed when PrimPol or RFWD3 are disrupted in cells that have been manipulated to lack SLFN11 (FIGS. 11B-D).

FIGS. 12A-C depict the results of HAP1 knockout cells transduced with lentivirus encoding full-length SLFN11 complementary DNA (cDNA) under the control of a doxycycline-inducible promoter. In the presence of doxycycline, SLFN11 genetic knockout cells reconstituted with pLIX403-SLFN11 have the same fork restart defect as observed in the parental cell line (FIGS. 12A-B), express SLFN11 protein at comparable levels to the parental HAP1 cells, and show reduced levels of ubiquitinated RPA32 (FIG. 12C).

FIGS. 13A-C depict the results of HAP1 SLFN11/RFWD3 or SLFN11/PRIMPOL knockout cells transduced with lentivirus encoding doxycycline inducible shRNAs. In the presence of doxycycline, the expression of the indicated proteins is suppressed as measured by western blot (FIG. 13A). Sensitivity to cisplatin is partially restored in SLFN11 KO cells when either RFWD3 (FIG. 13B) or PRIMPOL (FIG. 13C) is not expressed.

### DETAILED DESCRIPTION

Cancer cells may respond to chemotherapeutic agents by undergoing replication stress. Replication stress has been defined as a transient stalling of the replisome during DNA replication. The effects of replication fork stalling can be chronic or sudden, leading to replication catastrophe. For example, replication stress may result from lesions on DNA caused by DNA damaging chemotherapy. Pausing of the replisome results in accumulation of single-stranded DNA (ssDNA) at stalled forks, which may be sensed by Replication Protein A (RPA). RPA serves multiple purposes, including protecting ssDNA from endogenous nucleases while also acting as a signaling hub for replication stress and DNA repair sensing. Cells may mitigate replication stress through conserved signaling pathways that sense stalled forks, recruit cellular machinery to stabilize and repair these forks, and limit the generation of additional replication stress by suppressing late origin firing. When cancer cells mitigate such replication stress, they may exhibit chemoresistance.

Schlafen family member 11 (SLFN11) is a predictive marker of sensitivity to multiple classes of DNA damaging agents in pan-cancer. Forward genetic screens identify SLFN11 as the top mediator of replication stress and acquired chemoresistance for different cell lines, physiological contexts, and perturbations. Cells that express SLFN11 are particularly sensitive to drugs that induce double-strand breaks and interstrand crosslinks by multiple mechanisms. SLFN11 may be a predictive biomarker of clinical chemotherapy response.

Without being bound by theory, the present disclosure provides a model by which SLFN11 sensitizes cancer cells to multiple classes of chemotherapeutics by antagonizing replication fork restart. In the model, SLFN11 antagonizes ringer finger and WD repeat domain (RFWD3), a mediator of replication fork restart and interstrand crosslink repair, resulting in a loss of ubiquitination of its canonical target, RPA 32 kDa subunit (RPA32), and potentially other replication fork components. This is a previously unknown mechanism by which cancer cells can become resistant to replication stress and DNA damage. This effect can be mediated through a loss or decrease of activity of a primase and DNA-directed polymerase (PrimPol), as measured by DNA fiber assay. Knocking down either RFWD3 or PrimPol in SLFN11 knockout cell lines or cancer cell lines that do not natively express SLFN11 protein by RNA interference rescues the fork restart defect observed in cancer cell lines that express SLFN11, restoring chemosensitivity. Thus, targeting RFWD3, PrimPol, or a combination thereof in human cancers can sensitize cancer cells to a broad-spectrum chemotherapeutics (including already approved drugs) whose mechanisms of action converge on DNA replication and DNA replication stress, whether they are de novo or acquired chemoresistant cancers. In some embodiments, decreasing a level of expression of RFWD3 or PrimPol may resensitize one or more cancer cells to a chemotherapy. In some embodiments, decreasing a level of RDWD3 or PrimPol protein may resensitize one or more cancer cells to a chemotherapy.

These strategies may also have utility in SLFN11-expressing (including normal or high-expressing) cancers to suppress the emergence of pre-existing or therapy-induced SLFN11-negative clones, a known mechanism of acquired chemoresistance. Taken together, targeting RFWD3 and/or PrimPol may have utility in any tumor, cancerous lesion, or hyperplastic condition in conjunction with DNA-targeted chemotherapy.

The present disclosure provides methods for treating cancer in a subject in need thereof or sensitizing a plurality of cancer cells to a chemotherapeutic agent. The cancer may be associated with a low level of SLFN11. The chemotherapeutic agent may damage a portion of DNA in the cancer cell, thereby causing replication stress.

In some embodiments, a cancerous cell may have a low level of SLFN11 transcription factor as compared to a noncancerous cell. In some embodiments, the method or composition may comprise decreasing an amount of an endogenous ubiquitin ligase in a cancer cell of a subject. In some embodiments, the method or composition may comprise decreasing a ligase activity of the endogenous ubiquitin ligase in a cancerous cell of a subject. In some embodiments the endogenous ubiquitin ligase has at least 80% sequence identity to SEQ ID NO: 1. In some embodiments the endogenous ubiquitin ligase has at least 80% sequence identity to SEQ ID NO: 2. Methods as disclosed herein may comprise sensitizing a plurality of cancerous cells to a chemotherapeutic agent.

### SLFN11

SLFN11 is a ˜900 amino acid protein composed of several domains with distinct functions that include tRNA hydrolysis and putative helicase activity. Recently, the structure of SLFN11 in dimeric form was resolved, including the surprising finding that SLFN11 can bind to ssDNA. The precise mechanism by which SLFN11 sensitizes cancer cells to DNA damage (and the relative importance of each of these functional domains) has remained controversial. Chemoresistance may result from cancer cells' ability to evade chemotherapeutic effects at the molecular level.

The present disclosure provides methods of treating a cancer cell in a subject or of increasing a sensitivity of a plurality of cancer cells to a chemotherapeutic agent. In some embodiments, the cancer is associated with a reduced activity or expression of SFLN11. In some embodiments, the subject is identified as having reduced expression or activity of SFLN11. The reduced expression or activity may be determined by comparing a level of SFLN11 expression to a threshold value. A plurality of cancer cells as disclosed herein may exhibit less than about 5% SFLN11 positivity by immunochemistry. A plurality of cancer cells as disclosed herein may exhibit less than about 5%, less than about 4.5%, less than about 4%, less than about 3.5%, less than about 3%, less than about 2.5%, less than about 2%, less than about 1.5%, less than about 1%, less than about 0.9%, less than about 0.8%, less than about 0.7%, less than about 0.6%, less than about 0.5%, less than about 0.4%, less than about 0.3%, less than about 0.2%, or less than about 0.1% SFLN11 positivity by immunochemistry.

Methods as disclosed herein may comprise upregulating an activity of a gene or molecule that is regulated by an expression of SFLN11.

### Ring Finger and WD Repeat Domain (RFWD3)

Ring Finger and WD Repeat Domain 3 (RFWD3) is a ubiquitin ligase that promotes ubiquitylation of proteins on single-stranded DNA (ssDNA). The expression of RFWD3 is significant in repair and signaling factors in response to DNA damage. SLFN11 may antagonize RFWD3 activity. Thus, in cancer cells with a reduced activity or expression of SFLN11, there may also be increased activity of RFWD3, thereby promoting repair and signaling factors in response to DNA damage, e.g., chemotherapy-induced DNA damage. The cancer cell may exhibit unrestrained fork restart arising from high activity levels of RFWD3. It may further exhibit single-stranded DNA replication gaps subsequent to the fork restart.

The present disclosure provides methods of treating a cancer cell in a subject or of increasing a sensitivity of a plurality of cancer cells to a chemotherapeutic agent. In some embodiments, the cancer cell or subject is identified as having reduced expression or activity of SFLN11. The method may comprise decreasing an amount of an endogenous ubiquitin ligase. The ubiquitin ligase may be, for example, an RFWD3. The RFWD3 may have or comprise the sequence of SEQ ID NO: 1. The RFWD3 may have or comprise a sequence having at least about 70%, 75%, 80%, 85%, 90%, or 95% identity to the amino acid sequence of SEQ ID NO: 1.

### PrimPol

PrimPol is a eukaryotic protein with DNA polymerase and DNA primase activities. PrimPol is involved in DNA replication, and can replicate without an RNA primer. Specifically in the context of DNA damage, when replication forks stall, PrimPol can be recruited to replicate past sites of DNA damage.

The present disclosure provides methods of treating a cancer cell in a subject or of increasing a sensitivity of a plurality of cancer cells to a chemotherapeutic agent. In some embodiments, the cancer cell or subject is identified as having reduced expression or activity of SFLN11. The method may comprise decreasing an amount of an endogenous enzyme. The endogenous enzyme may have DNA polymerase and DNA primase activity. The endogenous enzyme may be PrimPol. The PrimPol may have or comprise the amino acid sequence of SEQ ID NO: 2. The PrimPol may have or comprise a sequence having at least about 70%, 75%, 80%, 85%, 90%, or 95% identity to the amino acid sequence of SEQ ID NO: 2.

TABLE 1
Sequences of Described Proteins

| SEQ ID NO | Sequence | Description |
|---|---|---|
| 1 | MAHEAMEYDVQVQLNHAEQQPAPAGMASSQGGPALLQPV | RFWD3 |
| | PADVVSSQGVPSILQPAPAEVISSQATPPLLQPAPQLSVDLTE | |
| | VEVLGEDTVENINPRTSEQHRQGSDGNHTIPASSLHSMTNFI | |
| | SGLQRLHGMLEFLRPSSSNHSVGPMRTRRRVSASRRARAGG | |
| | SQRTDSARLRAPLDAYFQVSRTQPDLPATTYDSETRNPVSEE | |
| | LQVSSSSDSDSDSSAEYGGVVDQAEESGAVILEEQLAGVSAE | |
| | QEVTCIDGGKTLPKQPSPQKSEPLLPSASMDEEEGDTCTICLE | |
| | QWTNAGDHRLSALRCGHLFGYRCISTWLKGQVRKCPQCNK | |
| | KARHSDIVVLYARTLRALDTSEQERMKSSLLKEQMLRKQAE | |
| | LESAQCRLQLQVLTDKCTRLQRRVQDLQKLTSHQSQNLQQP | |
| | RGSQAWVLSCSPSSQGQHKHKYHFQKTFTVSQAGNCRIMA | |
| | YCDALSCLVISQPSPQASFLPGFGVKMLSTANMKSSQYIPMH | |
| | GKQIRGLAFSSYLRGLLLSASLDNTIKLTSLETNTVVQTYNA | |
| | GRPVWSCCWCLDEANYIYAGLANGSILVYDVRNTSSHVQE | |
| | LVAQKARCPLVSLSYMPRAASAAFPYGGVLAGTLEDASFW | |
| | EQKMDFSHWPHVLPLEPGGCIDFQTENSSRHCLVTYRPDKN | |
| | HTTIRSVLMEMSYRLDDTGNPICSCQPVHTFFGGPTCKLLTK | |
| | NAIFQSPENDGNILVCTGDEAANSALLWDAASGSLLQDLQT | |
| | DQPVLDICPFEVNRNSYLATLTEKMVHIYKWE | |
| 2 | MNRKWEAKLKQIEERASHYERKPLSSVYRPRLSKPEEPPSIW | PrimPol |
| | RLFHRQAQAFNFVKSCKEDVHVFALECKVGDGQRIYLVTT | |
| | YAEFWFYYKSRKNLLHCYEVIPENAVCKLYFDLEFNKPANP | |
| | GADGKKMVALLIEYVCKALQELYGVNCSAEDVLNLDSSTD | |
| | EKFSRHLIFQLHDVAFKDNIHVGNFLRKILQPALDLLGSEDD | |
| | DSAPETTGHGFPHFSEAPARQGFSFNKMFTEKATEESWTSNS | |
| | KKLERLGSAEQSSPDLSFLVVKNNMGEKHLFVDLGVYTRN | |
| | RNFRLYKSSKIGKRVALEVTEDNKFFPIQSKDVSDEYQYFLS | |
| | SLVSNVRFSDTLRILTCEPSQNKQKGVGYFNSIGTSVETIEGF | |
| | QCSPYPEVDHFVLSLVNKDGIKGGIRRWNYFFPEELLVYDIC | |
| | KYRWCENIGRAHKSNNIMILVDLKNEVWYQKCHDPVCKAE | |
| | NFKSDCFPLPAEVCLLFLFKEEEEFTTDEADETRSNETQNPH | |
| | KPSPSRLSTGASADAVWDNGIDDAYFLEATEDAELAEAAEN | |
| | SLLSYNSEVDEIPDELIIEVLQE | |

### Methods of Treating a SLFN11-Low Cancer

The present disclosure provides methods of treating a cancer or of sensitizing a plurality of cancerous cells to a chemotherapeutic agent. Low-level expression of SFLN11 can cause cancer cells to leverage endogenous machinery to repair chemotherapy-induced DNA damage. For example, the cancer cells may exhibit unrestrained fork restart. They may further exhibit single-stranded DNA replication gaps subsequent to the fork restart. Methods as disclosed herein may comprise treating a cancer that is associated with a reduced activity or expression of SFLN11. In some embodiments, a subject having cancer is identified as having reduced expression or activity of SFLN11. The reduced expression or activity may be determined by comparing a level of SFLN11 expression to a threshold value. A plurality of cancer cells as disclosed herein may exhibit less than about 5% SFLN11 positivity by immunochemistry. A plurality of cancer cells as disclosed herein may exhibit less than about 5%, less than about 4.5%, less than about 4%, less than about 3.5%, less than about 3%, less than about 2.5%, less than about 2%, less than about 1.5%, less than about 1%, less than about 0.9%, less than about 0.8%, less than about 0.7%, less than about 0.6%, less than about 0.5%, less than about 0.4%, less than about 0.3%, less than about 0.2%, or less than about 0.1% SFLN11 positivity by immunochemistry.

The terms “treat” or “treatment” of a state, disorder or condition as described herein include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or sub-clinical symptoms of the state, disorder or condition; or (2) inhibiting the state, disorder or condition, i.e., arresting, reducing, delaying or reversing the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

As used herein, the term “therapeutically effective amount” refers to the amount of an agent (e.g., a chemotherapeutic agent) or a composition that, when administered to a subject for treating (e.g., preventing, ameliorating, or reversing) a state, disorder or condition, is sufficient to effect such treatment. The “therapeutically effective amount” will vary depending, e.g., on the compound, or analogues administered as well as the disease, its severity, and physical conditions and responsiveness of the subject to be treated.

The terms “subject”, “individual”, “patient”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

Methods as disclosed herein may comprise reducing an amount of an endogenous ubiquitin ligase in a plurality of cells. Methods as disclosed herein may comprise reducing the ligase activity of the endogenous ubiquitin ligase in cancerous cells of the subject. Methods as disclosed herein may comprise reducing both an amount of an endogenous ubiquitin ligase in a plurality of cells and ligase activity of the endogenous ubiquitin ligase in cancerous cells of the subject. The endogenous ubiquitin ligase may be, for example, RFWD3. The RFWD3 may have (or comprise a sequence having) at least about 70%, 75%, 80%, 85%, 90%, 95% sequence identity to SEQ ID NO: 1.

The gene expression of the ubiquitin ligase may be decreased or knocked out. For example, the gene may be knocked out via an engineered DNA nuclease such as CRISPR-associated protein (Cas) nucleases, transcription activator-like effector nucleases (TALEN), zinc-finger nuclease (ZFNs), meganucleases, other endo- or exo-nucleases, variants thereof, fragments thereof, and combinations thereof. The gene may be knocked down via any suitable method, such as through use of one or more anti-sense oligonucleotides (ASOs). The decreasing the amount of the endogenous ubiquitin ligase may comprise a post-transcriptional gene silencing (PTSGS) or RNA interference (RNAi). The gene expression of the ubiquitin ligase may be decreased by delivering a microRNA (miRNA) or a precursor thereof to a subject. The miRNA may be configured to bind to an mRNA encoding the endogenous ubiquitin ligase.

The ligase activity of the endogenous ubiquitin ligase may be reduced or inhibited by an inhibitor, such as but not limited to, a small molecule, an antibody or antibody fragment, a peptide, a polypeptide, a peptide analog, a fusion peptide, a polynucleotide, a small interfering RNA (siRNA), a short hairpin RNA (shRNA), a miRNA, an antisense oligonucleotide (ASO), a peptidomimetic, a natural product, a carbohydrate, an aptamer, an avimer, an anticalin, or a speigelmer.

Methods as disclosed herein may comprise reducing an amount of an endogenous enzyme in a plurality of cells. The endogenous enzyme may have DNA polymerase and DNA primase activity. Methods as disclosed herein may comprise reducing the primase and/or polymerase activity of the endogenous enzyme having DNA polymerase and DNA primase activity in cancerous cells. Methods as disclosed herein may comprise reducing both an amount of an endogenous enzyme having DNA polymerase and DNA primase activity and the primase and/or polymerase activity of the endogenous enzyme having DNA polymerase and DNA primase activity in cancerous cells. The endogenous enzyme may be PrimPol. The PrimPol may have or comprise the amino acid sequence of SEQ ID NO: 2. The PrimPol may have or comprise a sequence having at least about 70%, 75%, 80%, 85%, 90%, or 95% identity to the amino acid sequence of SEQ ID NO: 2.

The gene expression of the endogenous enzyme (e.g., PrimPol) may be decreased or knocked out. For example, the gene may be knocked out via an engineered DNA nuclease such as CRISPR-associated protein (Cas) nucleases, transcription activator-like effector nucleases (TALEN), zinc-finger nuclease (ZFNs), meganucleases, other endo- or exo-nucleases, variants thereof, fragments thereof, and combinations thereof. The gene may be knocked down via any suitable method, such as through use of one or more anti-sense oligonucleotides (ASOs). The decreasing the amount of the endogenous enzyme (e.g., PrimPol) may comprise a post-transcriptional gene silencing (PTSGS) or RNA interference (RNAi). The gene expression of the endogenous enzyme (e.g., PrimPol) may be decreased by delivering a microRNA (miRNA) or a precursor thereof to a subject. The miRNA may be configured to bind to an mRNA encoding the endogenous enzyme (e.g., PrimPol).

The primase and/or polymerase activity of the endogenous enzyme having DNA polymerase and DNA primase activity may be reduced or inhibited by an inhibitor, such as but not limited to, a small molecule, an antibody or antibody fragment, a peptide, a polypeptide, a peptide analog, a fusion peptide, a polynucleotide, a small interfering RNA (siRNA), a short hairpin RNA (shRNA), a miRNA, an antisense oligonucleotide (ASO), a peptidomimetic, a natural product, a carbohydrate, an aptamer, an avimer, an anticalin, or a speigelmer.

In some embodiments, the gene expression of an endogenous ubiquitin ligase and/or an endogenous enzyme (e.g., PrimPol) may be reduced by delivering a gene editing system. Gene editing system that may be used in the methods of the present disclosure include, but are not limited to, CRISPR/Cas systems, zinc finger nucleases (ZFNs), transcription activator-like effector nucleases (TALENs), as well as approaches using meganucleases, restriction endonucleases, or recombinases. In general, these tools edit a cellular genome by introducing a double-strand break (DSB) or a single-strand nick at a defined DNA target. Targeted cleavage or nicking is achieved with sequence-specific nucleases (e.g., engineered ZFNs, TALENs) or with CRISPR/Cas directed by a guide RNA (gRNA). Gene disruption (knockout/knockdown) can be accomplished by deleting or otherwise altering a target gene, for example via point mutation, insertion, deletion, frameshift, or missense mutations. Alternatively, gene insertion (knock-in) or gene replacement can be performed by supplying a donor template bearing an exogenous sequence and flanking homology arms corresponding to the genomic locus targeted by the gene editing system.

In some embodiments, gene expression of an endogenous ubiquitin ligase and/or an endogenous enzyme (e.g., PrimPol) may be reduced by delivering a small interfering RNA (siRNA). siRNAs are also known as short interfering RNAs or silencing RNAs. siRNAs are a class of double-stranded RNA molecules, typically about 20-25 base pairs in length that target nucleic acids (e.g., mRNAs) for degradation via the RNA interference (RNAi) pathway in cells. Such siRNA molecules typically include a region of sufficient homology to the target region, and are of sufficient length in terms of nucleotides, such that the siRNA molecules down-regulate target nucleic acids. It is not necessary that there be perfect complementarity between the siRNA molecule and the target, but the correspondence must be sufficient to enable the siRNA molecule to direct sequence-specific silencing, such as by RNAi cleavage of the target RNA. In some embodiments, the sense strand needs only to be sufficiently complementary with the antisense strand to maintain the overall double-stranded character of the molecule.

In some embodiments, gene expression of an endogenous ubiquitin ligase and/or an endogenous enzyme (e.g., PrimPol) may be reduced by delivering a short hairpin RNA (shRNA). A “small hairpin RNA” or “short hairpin RNA” or “shRNA” described herein may include a short RNA sequence that makes a tight hairpin turn that can be used to silence gene expression via RNA interference. The shRNAs provided herein may be chemically synthesized or transcribed from a transcriptional cassette in a DNA plasmid. The shRNA hairpin structure may be cleaved by the cellular machinery into siRNA, which is then bound to the RNA-induced silencing complex (RISC).

Non-limiting examples of shRNAs include a double-stranded polynucleotide molecule assembled from a single-stranded molecule, where the sense and antisense regions are linked by a nucleic acid-based or non-nucleic acid-based linker; and a double-stranded polynucleotide molecule with a hairpin secondary structure having self-complementary sense and antisense regions. In some embodiments, the sense and antisense strands of the shRNA are linked by a loop structure comprising from about 1 to about 25 nucleotides, from about 2 to about 20 nucleotides, from about 4 to about 15 nucleotides, from about 5 to about 12 nucleotides, or 1, 2, 3, 4, 5, 6, 7, 8, 9, 10, 11, 12, 13, 14, 15, 16, 17, 18, 19, 20, 21, 22, 23, 24, 25, or more nucleotides.

In some embodiments, the siRNAs or shRNAs of the present disclosure comprise an ortholog version (e.g., human) of the sequences described herein.

Specificity of siRNA molecules may be measured via the binding of the antisense strand of the molecule to its target RNA. Effective siRNA molecules are often fewer than 30 to 35 base pairs in length, e.g., to prevent stimulation of non-specific RNA interference pathways in the cell by way of the interferon response, however longer siRNA may also be effective. In various embodiments, the siRNA molecules are 5, 6, 7, 8, 9, 10, 11, 12, 13, 14, 15, 16, 17, 18, 19, 20, 21, 22, 23, 24, 25, 26, 27, 28, 29, or 30 base pairs in length. In various embodiments, the siRNA molecules are about 35 to about 70 more base pairs in length. In some embodiments, the siRNA molecules are more than 70 base pairs in length. In some embodiments, the siRNA molecules are 8 to 40 base pairs in length, 10 to 20 base pairs in length, 10 to 30 base pairs in length, 15 to 20 base pairs in length, 19 to 23 base pairs in length, 21 to 24 base pairs in length. In some embodiments, the sense and antisense strands of the siRNA molecules are each independently about 19 to about 24 nucleotides in length. In some embodiments, the sense strand of an siRNA molecule is 23 nucleotides in length and the antisense strand is 21 nucleotides in length. In some embodiments, both the sense strand and the antisense strand of an siRNA molecule are 21 nucleotides in length.

After selection of a suitable target RNA sequence, siRNA molecules that comprise a nucleotide sequence complementary to all or a portion of the target sequence, i.e., an antisense sequence, may be designed and prepared using suitable methods (see, e.g., U.S. Patent Publication Nos. 2004/0077574 and 2008/0081791 and PCT Publication No. WO 2004/016735). In some embodiments, the siRNA molecule may be single-stranded (i.e., a ssRNA molecule comprising just an antisense strand) or double stranded (i.e., a dsRNA molecule comprising an antisense strand and a complementary sense strand that hybridizes to form the dsRNA). In various embodiments, the siRNA molecules may comprise a duplex, asymmetric duplex, hairpin or asymmetric hairpin secondary structure, comprising self-complementary sense and/or antisense strands.

In some embodiments, the antisense strand of the siRNA molecule is 5, 6, 7, 8, 9, 10, 11, 12, 13, 14, 15, 16, 17, 18, 19, 20, 21, 22, 23, 24, 25, 26, 27, 28, 29, or 30 nucleotides in length. In various embodiment, the antisense strand of the siRNA molecule is about 35 to about 70 nucleotides in length. In various embodiment, the antisense strand of the siRNA molecule is more than 70 nucleotides in length. In some embodiments, the antisense strand is 8 to 40 nucleotides in length, 10 to 20 nucleotides in length, 10 to 30 nucleotides in length, 15 to 20 nucleotides in length, 19 to 23 nucleotides in length, or 21 to 24 nucleotides in length.

In some embodiments, the sense strand of the siRNA molecule is 5, 6, 7, 8, 9, 10, 11, 12, 13, 14, 15, 16, 17, 18, 19, 20, 21, 22, 23, 24, 25, 26, 27, 28, 29, or 30 more nucleotides in length. In various embodiments, the sense strand of the siRNA molecule is about 30 to about 70 nucleotides in length. In various embodiments, the sense strand of the siRNA molecule more than 70 nucleotides in length. In some embodiments, the sense strand is 8 to 40 nucleotides in length, 10 to 20 nucleotides in length, 10 to 30 nucleotides in length, 15 to 20 nucleotides in length, 19 to 23 nucleotides in length, 21 to 24 nucleotides in length.

In various embodiments, siRNA molecules can comprise an antisense strand comprising a region of complementarity to a target region in a target mRNA. In some embodiments, the region of complementarity is at least 70%, at least 75%, at least 80%, at least 85%, at least 90%, at least 91%, at least 92%, at least 93%, at least 94%, at least 95%, at least 96%, at least 97%, at least 98%, at least 99% or 100% complementary to a target region in a target mRNA. In some embodiments, the target region may comprise a region of consecutive nucleotides in the target mRNA. In some embodiments, it may not be requisite for a region of complementarity to be 100% complementary to that of its target to be specifically hybridizable or specific for a target RNA sequence.

In some embodiments, siRNA molecules disclosed herein may comprise an antisense strand that comprises a region of complementarity to a target RNA sequence and the region of complementarity is in the range of 8 to 20, 8 to 35, 8 to 45, or 10 to 50, or 5 to 55, or 5 to 40 nucleotides in length. In some embodiments, a region of complementarity is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary with at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, or more consecutive nucleotides of a target RNA sequence. In some embodiments, siRNA molecules comprise an antisense strand having a nucleotide sequence that contains no more than 1, 2, 3, 4, 5, 6, 7, 8, 9, or 10 base mismatches compared to the portion of the consecutive nucleotides of target RNA sequence. In some embodiments, siRNA molecules comprise a nucleotide sequence that has up to 3 mismatches over 15 bases, or up to 4 mismatches over 10 bases with a target sequence. In some embodiments, siRNA molecules comprise an antisense strand having a nucleotide sequence that has up 0, 1, 2, or 3 mismatches over 15-22 bases with a target sequence. In some embodiments, siRNA molecules comprise an antisense strand having a nucleotide sequence that has 0, 1, or 2 mismatches over 15-22 bases with a target sequence. In some embodiments, siRNA molecules comprise an antisense strand having a nucleotide sequence that has 0 or 1 mismatch over 15-22 bases with a target sequence. In some embodiments, siRNA molecules comprise an antisense strand having a nucleotide sequence that has 0 mismatches over 15-22 bases with a target sequence.

In various embodiments, siRNA molecules may comprise an antisense strand comprising a nucleotide sequence that is at least 70%, at least 75%, at least 85%, at least 90%, at least 95%, or 100% complementary to the target RNA sequence of the antisense oligonucleotides disclosed herein. In some embodiments, siRNA molecules comprise an antisense strand comprising a nucleotide sequence that is at least 70%, at least 75%, at least 85%, at least 90%, at least 95%, or 100% identical to any of the antisense oligonucleotides provided herein. In some embodiments, siRNA molecules comprise an antisense strand comprising at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, or more consecutive nucleotides of any of the antisense oligonucleotides provided herein.

In some embodiments, double-stranded siRNA can comprise sense and anti-sense RNA strands that are different lengths or the same length. In some embodiments, double-stranded siRNA molecules may also be generated from a single oligonucleotide in a stem-loop structure. The self-complementary sense and antisense regions of the siRNA molecule having a stem-loop structure may be linked by means of a nucleic acid based or a non-nucleic acid-based linker. In some embodiments, an siRNA having a stem-loop structure comprises a circular single-stranded RNA having two or more loop structures and a stem comprising self-complementary sense and antisense strands. In some embodiments, the circular RNA may be processed in vivo or in vitro to produce an active siRNA molecule which may be capable of mediating RNAi. Small hairpin RNA (shRNA) molecules are therefore also contemplated in the present disclosure. Such molecules may comprise a specific antisense sequence together with the reverse complement (sense) sequence, which may be separated by a spacer or loop sequence in some instances. A reverse complement described herein may comprise a sequence that is a complement sequence of a reference sequence, wherein the complement sequence is written in the reverse orientation. Due to codon usage redundancy, a reverse complement can diverge from a reference sequence that encodes the same polypeptide. As used herein, “reverse complement” also includes sequences that are, e.g., at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the reverse complement sequence of a reference sequence. Cleavage of the spacer or loop can provide a single-stranded RNA molecule and its reverse complement, such that they may anneal to form a dsRNA molecule. In various embodiments, additional optional processing steps may result in removal or addition of 1, 2, 3, 4, 5 or more nucleotides from the 3′ end and/or the 5′ end of one or both strands. A spacer may be of a suitable length to allow the antisense and sense sequences to anneal and form a double-stranded structure or stem prior to cleavage of the spacer. In certain embodiments subsequent optional processing steps may result in removal or addition of 1, 2, 3, 4, 5 or more nucleotides from the 3′ end and/or the 5′ end of one or both strands. In some embodiments, a spacer sequence can be an unrelated nucleotide sequence that may be, e.g., situated between two complementary nucleotide sequence regions that, when annealed into a double-stranded nucleic acid, can comprise a shRNA.

In various embodiments, an siRNA molecule can comprise a 3′ overhang at one end of the molecule. In some embodiments, the other end can be blunt-ended or may also comprise an overhang (e.g., 5′ and/or 3′). When the siRNA molecule comprises an overhang at both ends of the molecule, the length of the overhangs may be different or the same. In some embodiments, an siRNA molecule described herein may comprises 3′ overhangs of about 1 to about 3 nucleotides on both ends of the molecule. In some embodiments, the siRNA molecule comprises 3′ overhangs of about 1 to about 3 nucleotides on both the sense strand and the antisense strand. In some embodiments, the siRNA molecule comprises 3′ overhangs of about 1 to about 3 nucleotides on the antisense strand. In some embodiments, the siRNA molecule may comprise 3′ overhangs of about 1 to about 3 nucleotides on the sense strand.

In various embodiments, the siRNA molecule comprises one or more modified nucleotides (e.g., 1, 2, 3, 4, 5, 6, 7, 8, 9, 10, 11, 12, 13, 14, 15 or more). In some embodiments, all of the nucleotides of the sense strand and/or the antisense strand of the siRNA molecule are modified. In certain embodiments, the siRNA molecule can comprise one or more modified nucleotides and/or one or more modified internucleotide linkages. In some embodiments, the siRNA molecule may comprise modified internucleotide linkages at the first and second internucleoside linkages at the 5′ end of the siRNA molecule sense strand. In some embodiments, the siRNA molecule may comprise modified internucleotide linkages at the first and second internucleoside linkages at the 5′ and 3′ ends of the siRNA molecule antisense strand. In some embodiments, the siRNA molecule may comprise modified internucleotide linkages at the first and second internucleoside linkages at the 5′ end of the siRNA molecule sense strand and at the first and second internucleoside linkages at the 5′ and 3′ ends of the siRNA molecule antisense strand.

In some embodiments, the modified nucleotide may comprise a modified sugar moiety (e.g., a 2′ modified nucleotide). In some embodiments, the siRNA molecule can comprise one or more 2′ modified nucleotides, e.g., a 2′-deoxy, 2′-fluoro (2′-F), 2′-O-methyl(2′-O-Me), 2′-O-methoxyethyl (2′-MOE), 2′-O-aminopropyl(2′-O-AP), 2′-O-dimethylaminoethyl(2′-O-DMAOE), 2′-O-dimethylaminopropyl(2′-O-DMAP), 2′-O-dimethylaminoethyloxyethyl(2′-O-DMAEOE), or 2′-O—N-methylacetamido (2′-O-NMA). In various embodiments, each nucleotide of the siRNA molecule can a modified nucleotide (e.g., a 2′-modified nucleotide). In some embodiments, the siRNA molecule may comprise one or more phosphorodiamidate morpholinos. In some embodiments, each nucleotide of the siRNA molecule consists of a phosphorodiamidate morpholino.

In various embodiments, the siRNA molecule may comprise a phosphorothioate or other modified internucleotide linkage. In various embodiments, the siRNA molecule may comprise, e.g., a phosphorothioate internucleoside linkage(s). In some embodiments, the siRNA molecule may comprise a phosphorothioate internucleoside linkage(s) between two or more nucleotides. In some embodiments, the siRNA molecule may comprise a phosphorothioate internucleoside linkage(s) between all nucleotides. In some embodiments, the siRNA molecule may comprise modified internucleotide linkages at the first, second, and/or third internucleoside linkage at the 5′ or 3′ end of the siRNA molecule. In some embodiments, the siRNA molecule may comprise modified internucleotide linkages at the first and second internucleoside linkages at the 5′ and/or 3′ end of the siRNA molecule. In some embodiments, the siRNA molecule may comprise modified internucleotide linkages at the first and second internucleoside linkages at the 5′ end of the siRNA molecule sense strand. In some embodiments, the siRNA molecule may comprise modified internucleotide linkages at the first and second internucleoside linkages at the 5′ and 3′ ends of the siRNA molecule antisense strand. In some embodiments, the siRNA molecule may comprise modified internucleotide linkages at the first and second internucleoside linkages at the 5′ end of the siRNA molecule sense strand and at the first and second internucleoside linkages at the 5′ and 3′ ends of the siRNA molecule antisense strand. In some embodiments, the siRNA molecule may comprise modified internucleotide linkages at the first internucleoside linkage at the 5′ and 3′ ends of the siRNA molecule sense strand, at the first, second, and third internucleoside linkages at the 5′ end of the siRNA molecule antisense strand, and at the first internucleoside linkage at the 3′ end of the siRNA molecule antisense strand.

A “variant” of a molecule is a sequence that is substantially similar to the sequence of the referenced molecule. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis. Generally, nucleotide sequence variants of the disclosure will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the referenced nucleotide sequence.

In some embodiments, gene expression of an endogenous ubiquitin ligase and/or an endogenous enzyme (e.g., PrimPol) may be reduced by delivering an antisense oligonucleotide (ASO). An antisense oligonucleotide can down-regulate a target by, for example, inducing RNase H-mediated cleavage of the target RNA, sterically blocking ribosomal activity, inhibiting 5′-cap formation, or modulating splicing. The ASO can be, without limitation, a gapmer or a morpholino. Typically, an ASO comprises a short nucleotide sequence substantially complementary to a target sequence in a pre-mRNA, heterogeneous nuclear RNA (hnRNA), or mRNA. The degree of complementarity is preferably sufficient to form a stable double-stranded hybrid with the target RNA under physiological conditions. ASOs are often synthetic and chemically modified.

Antisense oligonucleotides may be fully complementary to the target sequence or may include intentional mismatches—e.g., to enhance selective targeting of an allele carrying a mutation-provided that the heteroduplex formed with the target remains sufficiently stable to withstand cellular nucleases and other in vivo degradation processes. Thus, in some embodiments, ASOs may have about or at least about 70% sequence complementarity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) to the target sequence. Backbone chemistries that are less susceptible to nuclease cleavage are also encompassed herein. When present, mismatches are typically less destabilizing near the duplex ends than in the central region. The allowable number of mismatches depends on oligonucleotide length, the G: C content of the duplex, and mismatch position, in accordance with established principles of duplex stability.

In some embodiments, an enzymatic activity of a target protein (e.g., ligase activity of the endogenous ubiquitin ligase, or primase and/or polymerase activity of an endogenous enzyme such as PrimPol) is reduced or inhibited by delivering an antibody or an antigen-binding fragment. In some embodiments, an antigen-binding fragment may be a bi-epitopic antibody or antigen-binding fragment thereof, a single-chain Fv (scFv), a Fab, a Fab′, a F(ab′)2, an Fv fragment, a disulfide stabilized Fv fragment (dsFv), a (dsFv)2, a VHH, a Fv-Fc fusion, a scFv-Fc fusion, a scFv-Fv fusion, a diabody, a tribody, or a tetrabody. In some embodiments, the antigen-binding fragment is an scFv. In some embodiments, the antigen-binding fragment is a Fab or Fab′. In some embodiments, the antibody is chimeric, human, partially humanized, fully humanized, or semi-synthetic. Antibodies and/or antibody fragments may be derived from murine antibodies, rabbit antibodies, human antibodies, fully humanized antibodies, camelid antibody variable domains and humanized versions, shark antibody variable domains and humanized versions, and camelized antibody variable domains.

In some embodiments, an enzymatic activity of a target protein (e.g., ligase activity of the endogenous ubiquitin ligase, or primase and/or polymerase activity of an endogenous enzyme such as PrimPol) is reduced or inhibited by delivering a polypeptide. The inhibitory polypeptide may be about 50 to about 1000 amino acids in length, such as about 50-800, 50-500, 50-400, 50-300 or 50-200 amino acids in length. In some embodiments, the inhibitory polypeptide may be about 50 to about 100 amino acids, about 100 to about 150 amino acids, or about 150 amino acids to about 200 amino acids in length.

In some embodiments, the antibody, an antigen-binding fragment, or inhibitory polypeptide described herein may comprise or be attached to a stabilizing domain. The stabilizing domain can be any domain that stabilizes the inhibitory polypeptide (for example, extending half-life of the inhibitory polypeptide in vivo). In some embodiments, the stabilizing domain is an Fc domain.

In some embodiments, the antibody, an antigen-binding fragment, or inhibitory polypeptide described herein comprises an Fc fragment. In some embodiments, the Fc fragment is selected from the group consisting of Fc fragments from IgG, IgA, IgD, IgE, IgM, and combinations and hybrids thereof. In some embodiments, the Fc fragment is derived from a human IgG. In some embodiments, the Fc fragment comprises the Fc region of human IgG1, IgG2, IgG3, IgG4, or a combination or hybrid IgG.

Methods as described herein may be used to resensitize one or more cancer cells to a chemotherapeutic agent. The chemotherapeutic agent may induce DNA damage in an S phase of the cancerous cell. The chemotherapeutic agent may be, for example, a PARP inhibitor, a DNA alkylating agent, a DNA crosslinker, a topoisomerase I inhibitor, a topoisomerase II inhibitor, a polymerase inhibitor, a polymerase alpha inhibitor, a clastogen, an antimetabolite, or any combination thereof.

Non-limiting examples of PARP inhibitors include olaparib, niraparib, rucaparib, talazoparib, veliparib, pamiparib, and fuzuloparib. Non-limiting examples of DNA alkylating agents include cyclophosphamide, ifosfamide, melphalan, chlorambucil, dacarbazine, procarbazine, temozolomide, busulfan, carmustine (BCNU), lomustine (CCNU), bendamustine, and thiotepa. Non-limiting examples of DNA crosslinkers include cisplatin, carboplatin, oxaliplatin, mitomycin C, nitrogen mustards (e.g., mechlorethamine), treosulfan, and psoralen (with UVA). Non-limiting examples of topoisomerase I inhibitors include irinotecan, topotecan, camptothecin analogs (e.g., SN-38), and liposomal irinotecan. Non-limiting examples of topoisomerase II inhibitors include etoposide, teniposide, doxorubicin, daunorubicin, epirubicin, idarubicin, and mitoxantrone; non-limiting examples of polymerase inhibitors include cytarabine (Ara-C), gemcitabine, fludarabine, cladribine, and aphidicolin. Non-limiting examples of polymerase alpha inhibitors include aphidicolin and other selective DNA polymerase-α inhibitors. Non-limiting examples of clastogens include bleomycin, etoposide, doxorubicin, cisplatin, mitomycin C, and ionizing radiation (e.g., X-ray or γ-ray). Non-limiting examples of antimetabolites include antifolates such as methotrexate, pemetrexed, pralatrexate, and raltitrexed; pyrimidine analogs such as 5-fluorouracil (5-FU), capecitabine, cytarabine (Ara-C), gemcitabine, floxuridine, trifluridine/tipiracil (TAS-102), azacitidine, and decitabine; purine analogs such as 6-mercaptopurine (6-MP), 6-thioguanine (6-TG), fludarabine, cladribine, nelarabine, and pentostatin; and other agents commonly classified as antimetabolites, including hydroxyurea.

Methods as disclosed herein can be used to treat a cancer or to increase a sensitivity of cancer cells to a chemotherapeutic agent. The cancer cells may be, for example, small cell lung cancer cells or neuroendocrine tumor cells. The neuroendocrine tumor may be a high-grade neuroendocrine tumor. The cancer cells may be treated with a chemotherapeutic agent prior to any of the methods as disclosed herein.

In some embodiments, the method comprises administering to the subject a combination of one or more agents that decrease the amount of an endogenous ubiquitin ligase and/or ligase activity of the endogenous ubiquitin ligase in cancerous cells, and a chemotherapeutic agent. Two or more agents described herein may be administered sequentially, simultaneously, and/or concurrently.

In some embodiments, the method comprises administering to the subject a combination of one or more agents that decrease the amount of an endogenous enzyme (e.g., PrimPol) and/or primase and/or polymerase activity of the endogenous enzyme (e.g., PrimPol) in the cancerous cells, and a chemotherapeutic agent. Two or more agents described herein may be administered sequentially, simultaneously, and/or concurrently.

In some embodiments, it is envisioned that two or more combinations of the agents of the disclosure will be administered to the subject. It is believed that the agents may also be administered in combination with one or more additional therapeutic agents. This combination can allow separate, continuous or simultaneous administration with the other active ingredients of the above agents. This combination may be provided in the form of a pharmaceutical composition.

As used herein, the term “combination” is used by the combination agents as defined above dependently or independently, or by the use of different fixed combinations with different amounts of combination agents, i.e., simultaneously or at different times. The term also refers to a kit of compositions or parts that can be administered. The combination agents can then be administered, for example, simultaneously or staggered in time (i.e., at different times and at equal or different time intervals for any part of the kit). The ratio of the total amount of combination agents administered in a combination can vary, e.g., to address the needs of a subpopulation of patients to be treated or the needs of a single patient, and different needs are the age of the patient, it can be due to gender, weight, etc.

The term “simultaneous administration,” as used herein, means that a first agent and second agent in a combination therapy are administered with a time separation of no more than about 15 minutes, such as no more than about any of 10, 5, or 1 minutes. When the first and second agents are administered simultaneously, the first and second agents may be contained in the same composition (e.g., a composition comprising both a first and second agent) or in separate compositions (e.g., a first agent in one composition and a second agent is contained in another composition).

As used herein, the term “sequential administration” means that the first agent and second agent in a combination therapy are administered with a time separation of more than about 15 minutes, such as more than about any of 20, 30, 40, 50, 60, or more minutes. Either the first agent or the second agent may be administered first. The first and second agents are contained in separate compositions, which may be contained in the same or different packages or kits.

As used herein, the term “concurrent administration” means that the administration of the first agent and that of a second agent in a combination therapy overlap with each other.

In certain embodiments, an agent described herein (e.g., an agent that decreases the amount of an endogenous ubiquitin ligase and/or ligase activity of the endogenous ubiquitin ligase in cancerous cells, an agent that decreases the amount of an endogenous enzyme (e.g., PrimPol) and/or primase and/or polymerase activity of the endogenous enzyme (e.g., PrimPol), a chemotherapeutic agent (e.g., a PARP inhibitor, a DNA alkylating agent, a DNA crosslinker, a topoisomerase I inhibitor, a topoisomerase II inhibitor, a polymerase or polymerase alpha inhibitor, a clastogen, an antimetabolite, or any combination thereof)) is administered to a subject at a dosing frequency of about once daily, about twice daily, about three times daily, about four times daily, about every other day, about seven times a week, about six times a week, about five times a week, about four times a week, about twice a week, about once a week, about once every two weeks, about once every three weeks, about once a month, about once every five weeks, about once every six weeks, about once every seven weeks, about once every two months, about once every three months, or less frequently so long as an effective therapeutic response is achieved.

In certain embodiments, the chemotherapeutic agent (e.g., a PARP inhibitor, a DNA alkylating agent, a DNA crosslinker, a topoisomerase I inhibitor, a topoisomerase II inhibitor, a polymerase or polymerase alpha inhibitor, a clastogen, an antimetabolite, or any combination thereof) can be administered about 10 minutes to about 2 hours, about 15 minutes to about 1 hour, about 30 minutes to about 4 hours, about 1 hour to about 2 hours, about 1 hour to about 4 hours, about 1 hour to about 6 hours, about 2 hours to about 6 hours, about 2 hours to about 12 hours, about 2 hours to about 24 hours, about 2 hours to about 48 hours, about 2 hours to about 72 hours, about 2 hours to about 48 hours, about 2 hours to about 24 hours, about 2 hours to about 12 hours, about 2 hours to about 6 hours, about 2 hours to about 4 hours, about 1 hour to about 72 hours, about 1 hour to about 48 hours, about 1 hour to about 24 hours, about 1 hour to about 12 hours, about 1 hour to about 6 hours, about 1 hour to about 4 hours, about 1 hour to about 2 hours, about 6 hours to about 48 hours, about 6 hours to about 24 hours, about 12 hours to about 48 hours, about 24 hours to about 48 hours, about 24 hours to about 72 hours, about 24 hours to about 96 hours, about 48 hours to about 72 hours, about 48 hours to about 96 hours, about 72 hours to about 96 hours, about 72 hours to about 120 hours (about 3-5 days), about 96 hours to about 168 hours (about 4-7 days), about 1 day to about 3 days, about 2 days to about 7 days, about 1 week to about 2 weeks, about 2 weeks to about 4 weeks, or longer intervals, prior to and/or subsequent to administration of an agent that decreases the amount of an endogenous ubiquitin ligase and/or ligase activity of the endogenous ubiquitin ligase in cancerous cells, or an agent that decreases the amount of an endogenous enzyme (e.g., PrimPol) and/or primase and/or polymerase activity of the endogenous enzyme (e.g., PrimPol).

In certain embodiments, a therapeutically effective amount of an agent that decreases the amount of an endogenous ubiquitin ligase and/or ligase activity of the endogenous ubiquitin ligase in cancerous cells, or an agent that decreases the amount of an endogenous enzyme (e.g., PrimPol) and/or primase and/or polymerase activity of the endogenous enzyme (e.g., PrimPol) is administered to a subject in need thereof. The agent may be administered at a dose of about 0.0001-100 mg/kg per dose, such as about 0.001-50 mg/kg, 0.01-10 mg/kg, about 0.05-5 mg/kg, or 0.1-5 mg/kg. In some embodiments, the dose is selected from 0.001, 0.002, 0.005, 0.01, 0.02, 0.03, 0.05, 0.075, 0.1, 0.2, 0.3, 0.4, 0.5, 0.75, 1, 1.5, 2, 3, 4, 5, 7.5, 10, 15, 20, 25, 30, 40, or 50 mg/kg. In other embodiments, a fixed dose is used (e.g., about 0.1-200 mg per subject, such as 1, 5, 10, 25, 50, 100, or 150 mg per subject).

In some embodiments, a therapeutically effective amount of a chemotherapeutic agent (e.g., a PARP inhibitor, a DNA alkylating agent, a DNA crosslinker, a topoisomerase I inhibitor, a topoisomerase II inhibitor, a polymerase or polymerase alpha inhibitor, a clastogen, an antimetabolite, or any combination thereof) is administered to a subject in need thereof. The agent may be administered at about 0.05-20 mg/kg per dose (e.g., about 0.01-10 mg/kg, about 0.1-10 mg/kg, about 0.05-5 mg/kg, or 0.1-5 mg/kg), about 5-1,000 mg/m2 per dose (e.g., about 25-750 mg/m2 or about 50-500 mg/m2), or as a fixed oral dose of about 10-1,000 mg per administration (e.g., about 25, 50, 100, 150, 200, 300, 400, or 600 mg/day). Dosing can be single or repeated, for example daily, twice daily, three times weekly, weekly, biweekly, or on a cyclic schedule (e.g., days 1-5, 1 and 8, or 1, 8, and 15 of a 21- or 28-day cycle), including continuous daily dosing for orally available agents (such as PARP inhibitors) or intermittent dosing for IV agents (such as topoisomerase inhibitors, alkylators, and crosslinkers).

The route of administration of the one or more agents (e.g., an agent that decreases the amount of an endogenous ubiquitin ligase and/or ligase activity of the endogenous ubiquitin ligase in cancerous cells, an agent that decreases the amount of an endogenous enzyme (e.g., PrimPol) and/or primase and/or polymerase activity of the endogenous enzyme (e.g., PrimPol), a chemotherapeutic agent (e.g., a PARP inhibitor, a DNA alkylating agent, a DNA crosslinker, a topoisomerase I inhibitor, a topoisomerase II inhibitor, a polymerase or polymerase alpha inhibitor, a clastogen, an antimetabolite, or any combination thereof)) can be by any suitable route including, but not limited to, oral, parenteral, intrathecal, intra-arterial, intraperitoneal, intravenous, subcutaneous, topical, intracranial, intratumoral, intranasal, or intramuscular. Systemic modes of administration include, but are not limited to, parenteral and oral routes. Parenteral routes include, but are not limited to, subcutaneous, intraosseous, intravenous, intranasal, intradermal, intraarterial, intramuscular, and intraperitoneal routes. Some specific examples are intravenous infusion, nasal instillation, and intravitreal injection. Local modes of administration include, but are not limited to, intracerebroventricular, intrathecal, intraparenchymal (e.g., localized intraparenchymal delivery to the cerebral cortex, temporal cortex, striatum, tegmentum, precentral gyrus, hippocampus, thalamus, frontal cortex, hypothalamus, cerebellum, amygdala, medulla, tectum, or substantia nigra), intravitreal, subconjunctival, intraocular, subretinal, intraorbital, and transscleral routes. Significantly smaller amounts of the active components may elicit an effect(s) when administered locally (e.g., intravitreal or intraparenchymal) compared to when administered systemically (e.g., intravenously). Administration locally may also reduce or eliminate the prevalence of potentially toxic side effects that may occur when the component(s) are administered systemically.

Administration in vivo can be by any suitable route including, but not limited to, intravenous, intracranial, subcutaneous, parenteral, intratumoral, intraperitoneal, oral, intra-arterial, intranasal, intrathecal, topical, intramuscular, lipid nanoparticle-based delivery (LNP), cellular delivery, viral and/or non-viral delivery or by any other genetic modifications or as a cargo in a cell.

The route of administration and the type of pharmaceutically acceptable carrier will depend on the condition being treated and the type of mammal. Formulations containing the active compound may be prepared such that the activity of the compound is not disrupted during the process and the compound can reach its site of action without disruption. In some cases, it may be necessary to protect the compound by means known in the art, such as microencapsulation. Similarly, the route of dosing selected should be such that the compound reaches its site of action.

Any of the agents described herein (e.g., an agent that decreases the amount of an endogenous ubiquitin ligase and/or ligase activity of the endogenous ubiquitin ligase in cancerous cells, an agent that decreases the amount of an endogenous enzyme (e.g., PrimPol) and/or primase and/or polymerase activity of the endogenous enzyme (e.g., PrimPol), a chemotherapeutic agent) can be present in a composition (such as a formulation) that includes other agents, excipients, or stabilizers.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

As used herein, “pharmaceutically acceptable carrier” or “pharmaceutical acceptable excipient” includes any material which, when combined with an active ingredient, allows the ingredient to retain biological activity and is non-reactive with the subject's immune system. Compositions comprising such carriers are formulated by well-known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

If the compound contains one or more functional groups that can be protonated or deprotonated (e.g., at physiological pH), the compound can be prepared and/or isolated as a pharmaceutically acceptable salt. It will be appreciated that the compound can be zwitterion at a given pH. As used herein, the expression “pharmaceutically acceptable salt” refers to a salt of a given compound, which salt is suitable for pharmaceutical administration. Such salts can be formed, for example, by reacting an acid or base with an amine or carboxylic acid group, respectively.

Pharmaceutically acceptable acid addition salts can be prepared from inorganic and organic acids. Examples of inorganic acids include hydrochloric acid, hydrobromic acid, sulfuric acid, nitric acid, phosphoric acid and the like. Examples of organic acids include acetic acid, propionic acid, glycolic acid, pyruvate, oxalic acid, malic acid, malonic acid, succinic acid, maleic acid, fumaric acid, tartrate acid, citrate, benzoic acid, cinnamic acid, mandelic acid, Examples thereof include methanesulfonic acid, ethanesulfonic acid, p-toluenesulfonic acid and salicylic acid.

Pharmaceutically acceptable base addition salts can be prepared from inorganic and organic bases. Corresponding counterions derived from inorganic bases include salts of sodium, potassium, lithium, ammonium, calcium and magnesium. Organic bases include isopropylamine, trimethylamine, diethylamine, triethylamine, tripropylamine, ethanolamine, 2-dimethylaminoethanol, tromethamine, lysine, arginine, histidine, caffeine, prokine, hydrabamine, choline, betaine, ethylenediamine, glucosamine, Substituted amines such as primary, secondary and tertiary amines such as N-alkylglucamine, theobromine, purines, piperazine, piperazine and N-ethylpiperidine, substituted amines such as natural substituted amines and cyclic amines can be mentioned.

Acid/base addition salts tend to be more soluble in aqueous solvents than the corresponding free acid/base forms.

The formulation may include suitable excipients, such as antioxidants. Examples of antioxidants include phenolic compounds such as BHT or Vitamin E, reducing agents such as methionine or sulfites, and metal chelating agents such as EDTA.

The compounds or pharmaceutically acceptable salts thereof described herein can be prepared in parenteral dosage forms such as those suitable for, e.g., intravenous, intrathecal, and intracerebral or epidural delivery. Suitable pharmaceutical forms for injectable use include sterile injectable or dispersions and sterile powders for the immediate preparation of sterile injectable solutions. They must be stable under manufacturing and storage conditions and protected from reduction or oxidation and the contaminating effects of microorganisms such as bacteria or fungi.

The solvent or dispersion medium for the injectable solution or dispersion may include either conventional solvents or carrier systems for the active compound, e.g., water, ethanol, polyols (e.g., glycerol, propylene glycol). Liquid polyethylene glycol, etc., suitable mixtures thereof, and vegetable oils may be included. Proper fluidity can be maintained, for example, by the use of a coating such as lecithin, the maintenance of the required particle size in the case of dispersions, and the use of surfactants. Prevention of the action of microorganisms can be performed as needed by incorporating various antibacterial and antifungal agents such as parabens, chlorobutanol, phenol, sorbic acid, thimerosal and the like. In many cases, it may be preferable to include agents that regulate osmotic pressure, such as sugar or sodium chloride. Preferably, the injectable formulation is isotonic with blood. Sustained absorption of the injectable composition can be brought about by the use of agents that delay absorption (e.g., aluminum monostearate and gelatin) in the composition. In some embodiments, the administration of the compounds, preferably inhibitor is via systemic or local delivery. Suitable pharmaceutical forms for injection can be delivered by any suitable route, including but not limited to intravenous, intramuscular, intracerebral, intrathecal, epidural injection or infusion.

Sterilized injectable solutions are prepared by adding the required amount of the compounds of the disclosure to a suitable solvent containing various other components, such as those listed above, as needed, followed by filtration sterilization. Generally, dispersions are prepared by incorporating various sterile active ingredients into a sterile vehicle containing a basic dispersion medium and other required ingredients from those described above. For sterile powders for the preparation of sterile injectable solutions, the preferred method of preparation is vacuum drying or lyophilization of the pre-sterile filtered solution of the active ingredient plus any additional desired ingredients.

Other pharmaceutical forms include the oral and enteral formulations, where the active compound can be formulated with an inert diluent or an assimilated edible carrier, or encapsulated in hard or softshell gelatin capsules. The formulations can also be tableted, or it can be incorporated directly into diet foods. For oral therapeutic administration, the active compound is taken up with excipients and used in the form of ingestible tablets, buccal or sublingual tablets, troches, capsules, elixirs, suspensions, syrups, wafers, etc. The amount of active compound in such a therapeutically useful composition is such that an appropriate dose can be obtained.

Tablets, lozenges, pills, capsules, etc. may also contain the ingredients listed below: binders such as gum, acacia, corn starch or gelatin; excipients such as dicalcium phosphate; corn starch, Disintegrants such as potato starch, arginic acid; lubricants such as magnesium stearate; sweeteners such as sucrose, lactose or saccharin, or flavors such as peppermint, winter green oil, or cherry flavor may be added. If the dosage unit form is a capsule, it may contain a liquid carrier in addition to the above types of materials. Various other materials may be present as a coating or in other ways to alter the physical form of the dosage unit. For example, tablets, pills, or capsules can be coated with shellac, sugar, or both. The syrup or elixir may contain active compounds, sucrose as a sweetener, methyl and propylparabens as preservatives, pigments and flavors such as cherry or orange flavors. Of course, any substance used to prepare the dosage unit form must be pharmaceutically pure and substantially non-toxic in the amount used. In addition, the compounds of the disclosure may be incorporated into sustained release formulations and formulations comprising those that specifically deliver the active peptide to a particular region of the intestine.

Liquid formulations can also be administered enterally via the stomach or esophageal canal. The enteral preparation can be prepared in the form of a suppository by mixing with a suitable base such as an emulsifying base or a water-soluble base. It is possible, but not necessary, to administer the compound of the present disclosure topically, intranasally, intravaginally, intraocularly or the like.

The appropriate formulation for the agent(s) of the disclosure can be adjusted for pH. Buffer systems are routinely used to provide pH values in the desired range and include carboxylic acid buffers such as acetates, citrates, lactates and succinates. In some embodiments, the composition is formulated to have a pH range of about 4.5 to about 9.0, including for example pH ranges of about any of 5.0 to about 8.0, about 6.5 to about 7.5, and about 6.5 to about 7.0. In some embodiments, the pH of the composition is formulated to no less than about 6, including for example no less than about any of 6.5, 7, or 8 (such as about 8). The composition can also be made to be isotonic with blood by the addition of a suitable tonicity modifier, such as glycerol.

Pharmaceutically acceptable vehicles and/or diluents include any and all solvents, dispersion media, coatings, antibacterial and antifungal agents, isotonic and absorption retarders, and the like. The use of such vehicles and agents for pharmaceutically active substances is well known in the art. Its use in therapeutic compositions is intended unless any conventional vehicle or agent is incompatible with the active ingredient. Auxiliary active ingredients can also be incorporated into the composition.

It is particularly advantageous to formulate the composition in unit dosage form for ease of administration and uniformity of dosage. As used herein, a dosage unit form means a physically distinct unit suitable as a unit dosage for a mammalian subject to be treated; each unit is a required pharmaceutically acceptable vehicle. Contains a predetermined amount of active substance calculated to produce the desired therapeutic effect in connection with. Details of the novel dosage unit forms of the disclosure include (a) the unique properties of the active substance and the particular therapeutic effect to be achieved, and (b) physical health as disclosed in detail herein. It is determined by and directly dependent on the technology-specific limitations of the active substances formulated for the treatment of the disease in living subjects with impaired disease states.

As mentioned above, the main active ingredient may be formulated for convenient and effective administration in therapeutically effective amounts using a suitable pharmaceutically acceptable vehicle in the form of a dosage unit. The unit dosage form can contain, for example, the major active compound in an amount ranging from 0.25 μg to about 2000 mg. Expressed in proportion, the active compound may be present in a carrier of about 0.25 μg to about 2000 mg/mL. In the case of a composition containing an auxiliary active ingredient, the dose is determined with reference to the usual dosage and mode of administration of the ingredient.

In some embodiments, the composition is suitable for administration to a human. In some embodiments, the composition is suitable for administration to a mammal such as, in the veterinary context, domestic pets and agricultural animals. There are a wide variety of suitable formulations of the composition comprising the agents described herein. The following formulations and methods are merely exemplary and are in no way limiting. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl and propylhydroxybenzoates, talc, magnesium stearate, and mineral oil. In some embodiments, the composition discussed herein is present in a dry formulation (such as lyophilized composition). The formulations can additionally include lubricating agents, wetting agents, emulsifying and suspending agents, preserving agents, sweetening agents or flavoring agents.

Formulations suitable for parenteral administration include aqueous and non-aqueous, isotonic sterile injection solutions, which can contain antioxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### EXAMPLES

### Example 1: Studies on the Effects of RFWD3 and PrimPol

### Materials and Methods

### Tissue Culture

HAP1 cells (Haplogen, Gmbh) were cultured at 37° C. in IMDM media (Gibco) supplemented with 10% FBS (Atlantic Biologicals). HAP1 cells expressing pLIX403-SLFN11 were cultured in IMDM media with 10% tetracycline negative FBS and 1 μM puromycin; SLFN11 expression was induced by addition of 1 mg/ml doxycycline (Fisher Scientific) to media for 48 hours. U2OS cells (ATCC) were cultured in DMEM media (Gibco) supplemented with 10% FBS, 1% penicillin/streptomycin (Gibco) and 1% glutamine. TERT-immortalized RPE-1 cells (ATCC) were grown in DMEM/F12 (Gibco) with 10% FBS, 1% penicillin/streptomycin, and 0.25% Sodium Bicarbonate (Gibco).

### CRISPR Gene Disruption

HAP1 cells were electroporated with Cas9 ribonucleoprotein complexes targeting coding exons 1, 3, and 4 of Schlafen family member 11 (SLFN11) using the Neon Transfection System (Thermo Fisher Scientific). Cells were allowed to recover in complete medium and expand before they were single-cell sorted into 96-well tissue culture plates by fluorescence activated cell sorting. Multiple individual clones for each cell line were expanded into 6-well plates, reserving a portion for cryopreservation. The 6-well plates were screened for clones harboring frameshift indels in the SLFN11 coding sequence using PCR amplification and Sanger sequencing across the targeted locus. Loss of SLFN11 protein expression was confirmed by immunoblotting.

### Molecular Cloning

SLFN11 cDNA was cloned directly from parental HAP1 cells by polymerase chain reaction into the pDONR221 plasmid using BP Gateway cloning (Thermo Fisher). SLFN11 cDNA was subcloned from pDONR221 into the doxycycline inducible pLIX403 lentiviral transfer vector in a similar fashion using LR Gateway cloning. Correct cloning outcomes were confirmed by whole-plasmid sequencing (Plasmidsaurus).

### Lentivirus Production

pLIX403-SLFN11 transfer vector (3 μg) was transfected into HEK293T/17 cells in T25 flasks along with lentiviral packaging vectors psPAX2 (2 μg) and pMD2.G (1 μg) using 12 μg polyethylenimine. The following day media was changed and the cells were incubated a further 48 h. Lentiviral supernatants were collected and clarified by centrifugation at 300 g for 5′ followed by syringe filtration using 0.45 μm pore size (Millipore).

### Lentivirus Transduction and Cell Line Generation

Between 100 and 1 μL of clarified lentiviral supernatants were added to SLFN11 knockout cells in 6-well plates in the presence of 8 μg/mL polybrene. After 48 h, media was replaced with fresh media containing a final concentration of 1 μg/mL puromycin. To favor selection of cells with a single integrated provirus, the well with surviving cells to which the least amount of viral supernatant was added was expanded as a stable cell line.

### Cell Viability Assays

Cells were plated at a concentration of 1,000 cells/well in 96-well opaque cell culture plates. The following day, cells were treated with the indicated chemotherapeutic agents at the doses and dilution factors indicated in each figure with at least 4 replicates per condition. After 72 h, CellTiterGlo 2 luminescent viability reagent was added to each plate according to the manufacturer's instructions and subsequently luminescence was detected using a multimode plate reader in luminescence mode (BioTek Synergy H1). Raw luminescence values were normalized to blank and untreated wells using the Prism software package (GraphPad). For each condition, a 7-point dose-response curve was fit to estimate EC50 values.

siRNA Transfections

Transient siRNA transfections were performed using Lipofectamine RNAimax (Invitrogen) reagents according to the manufacturer's instructions. Downstream analyses were performed after 72 hr of siRNA knockdown.

### DNA Fiber Analysis

For analysis of replication fork restart, cells were pulse-labeled with 50 mM IdU for 20 min, washed twice with PBS, and treated with 2 mM hydroxyurea (HU) for 4 hr to stall replication through nucleotide depletion. Replication was then allowed to resume by washing out HU with PBS and adding back fresh media with 50 mM CIdU for 30 min. For experiments involving S1 nuclease digestion, following the CIdU pulse, cells were treated with CSK100 buffer (100 mM NaCl, 10 mM HEPES, 3 mM MgCl₂ [pH 7.2], 300 mM sucrose, and 0.5% Triton X-100) for 10 min at room temperature, then incubated with S1 nuclease buffer (30 mM sodium acetate [pH 4.6], 10 mM zinc acetate, 5% glycerol, and 50 mM NaCl) with or without 20 U/mL S1 nuclease (Thermo Scientific, EN0321) for 30 min at 37° C. Cells were then collected, washed and resuspended in cold 1×PBS at a density of 1×10⁶ cells/ml before spotting on a glass slide. Cells were lysed in SDS lysis buffer [0.5% SDS, 200 mM Tris-HCl (pH 7.4), 50 mM EDTA] for 6 minutes prior to tilting at a 15° angle to allow DNA spreading. Stretched DNA fibers were fixed in a chilled solution of methanol: acetic acid (3:1) for 3 minutes, denatured with 2.5 N HCl for 30 minutes, washed in 1×PBS, and incubated in blocking buffer (5% BSA in PBS with 0.1% Triton X-100) for 1 h at room temperature. Slides were probed with primary antibodies (mouse anti-IdU [B44] (BD Biosciences 347580, 1:150 dilution) and rat anti-CIdU [BU1/75 (ICR1)] (Abcam ab6326, 1:200 dilution)) and then with fluorescent-dye conjugated secondary antibodies (goat anti-mouse IgG (H+L) Alexa Fluor 488 (Thermo Fisher A11001, 1:350 dilution) and goat anti-rat IgG (H+L) Alexa Fluor 594 (Thermo Fisher A11007, 1:350 dilution)). Images were collected using a Keyence BZ-X710 microscope. Fork speed following restart (CIdU tract length) was assessed in a minimum of 180 fibers for each independent experiment, and the analysis shows the pool of three biological replicates per condition. A minimum of 250 DNA fibers were measured for each independent experiment of percentage of restart (% stalled forks), and analysis shows mean for three independent experiments. Tract lengths were determined in ImageJ using the scale of 1 μm=2.59 kb.

### Lysis Conditions and Immunoblotting

For routine western blotting analysis, cells were lysed in denaturing SDS buffer (100 mM Tris (pH 6.8), 2% SDS and 20 mM b-mercaptoethanol), and cell extracts were separated on NuPAGE 4-12% Bis-Tris or 3-8% Tris-Acetate gels (Invitrogen). Proteins were transferred onto 0.45 mm PVDF membrane (Millipore) in Tris-Glycine transfer buffer (Invitrogen). Membranes were blocked in 5% milk in TBS-T for 1 hr prior to incubation with primary antibody overnight. The following day, membranes were incubated with HRP-conjugated secondary antibodies (Peroxidase AffiniPure Goat Anti-Mouse IgG (H+L) (Jackson Labs 115-035-003, 1:10,000 dilution) and Peroxidase AffiniPure Goat Anti-Rabbit IgG (H+L) (Jackson Labs 111-035-003, 1:10,000 dilution)) in 5% milk in TBS-T and developed using ECL Prime reagent (GE Healthcare).

### Results

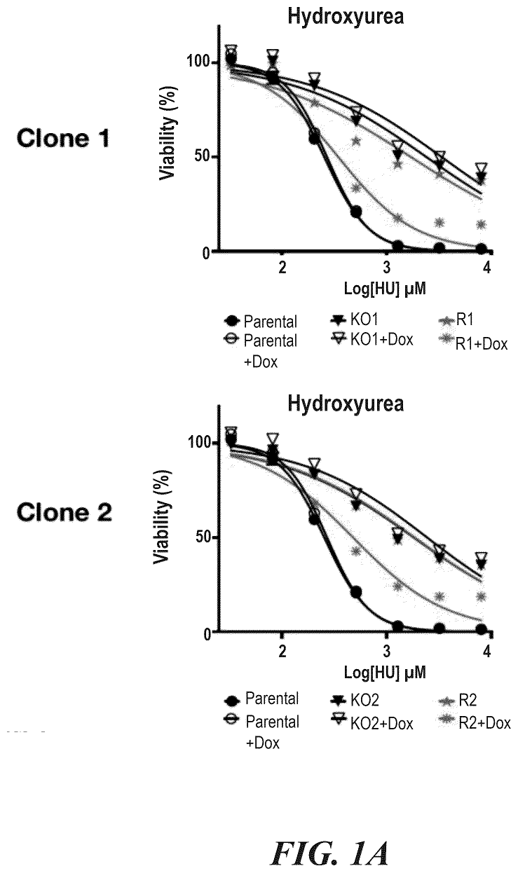

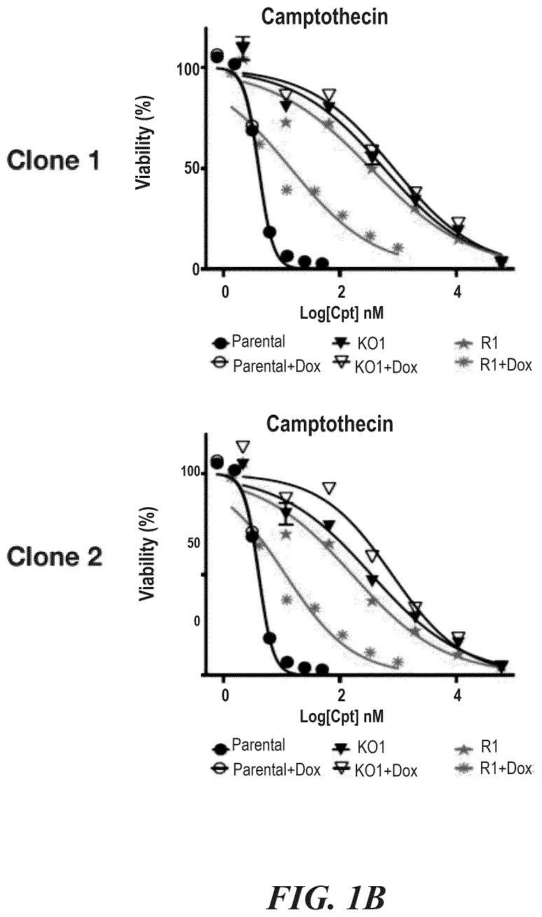

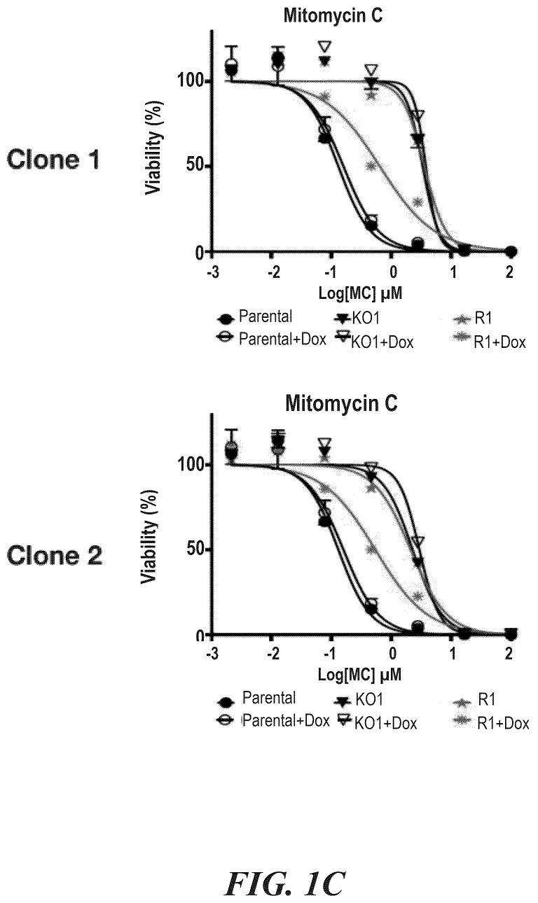

A HAP1 parental cell line, two SLFN11 knockout clones, and two knockout clones reconstituted with SLFN11 cDNA under the control of a doxycycline-inducible promoter were assessed for sensitivity to hydroxyurea and mitomycin C by 7-point dose-response curves in the presence or absence of doxycycline. Doxycycline induced an ˜5-fold increase in sensitivity to both drugs in the reconstituted SLFN11 knockout cell lines R1 and R2. Doxycycline caused no further increase in chemosensitivity in any other condition (FIG. 1A-C).

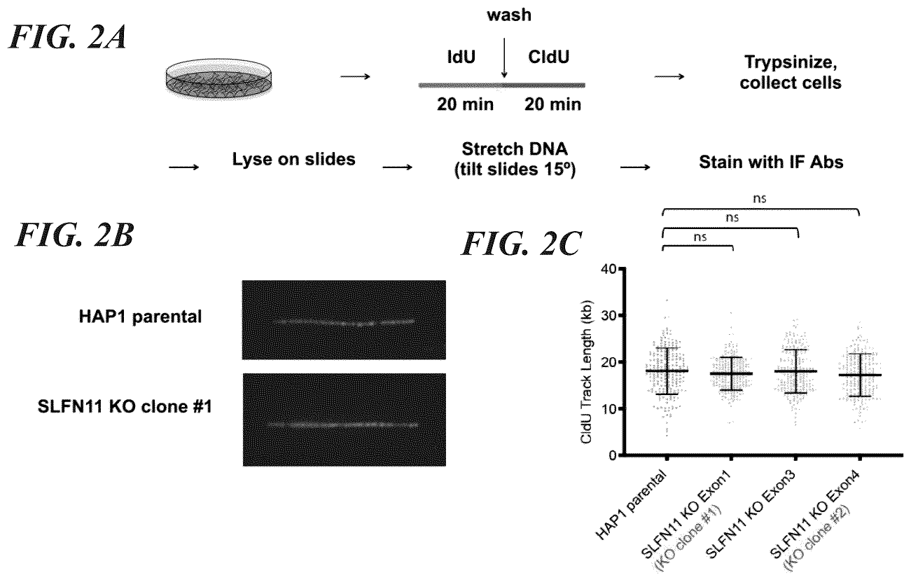

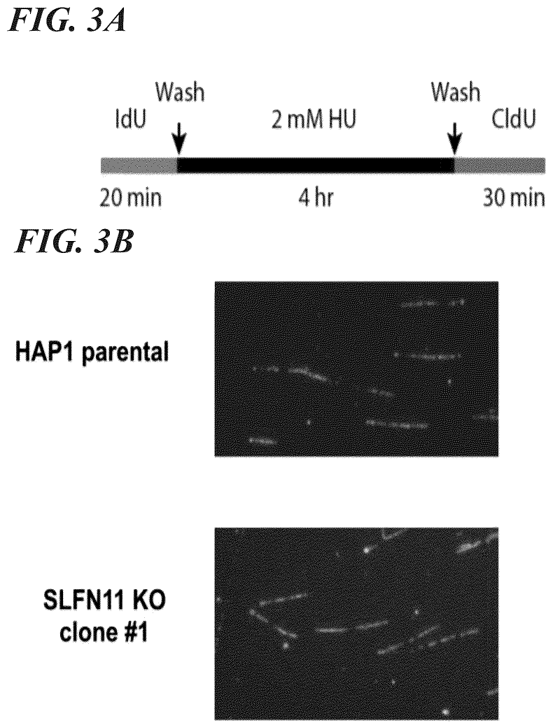

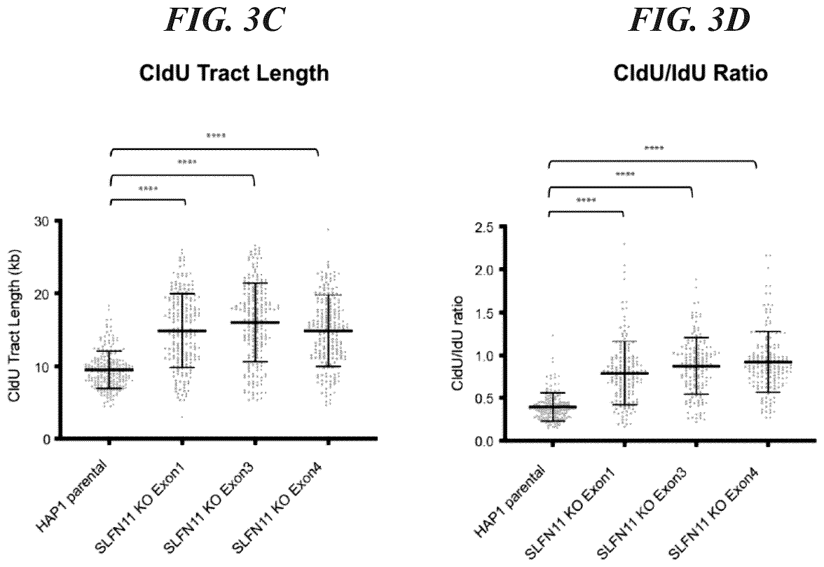

DNA fiber analysis, a single-molecule assay of DNA replication, was used to test fork speed in the context of SLFN11-expressing parental HAP1 cells or three different CRISPR knockout cell lines that were disrupted in exon 1, 3 or 4 of SLFN11 (FIG. 2A). The results of this assay demonstrated no difference in baseline replication rate in unperturbed conditions (FIG. 2B-C). It was next hypothesized that SLFN11 knockout cells might be more proficient in replication fork restart. A DNA fiber assay and a standard pulse-chase experiment were conducted in which incorporation of the CIdU thymidine analog after fork stalling is measured relative to IdU incorporation prior to fork stalling (FIGS. 3A-B). A striking increase was observed in absolute CIdU track length (FIG. 3C) and in relative CIdU tract length (FIG. 3D) in SLFN11 KO cells, consistent with accelerated fork progression following restart.

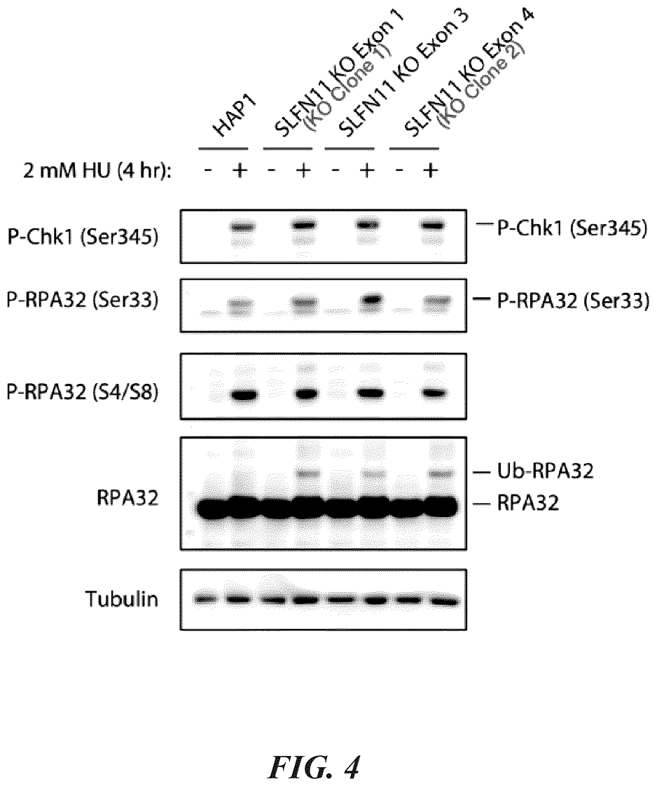

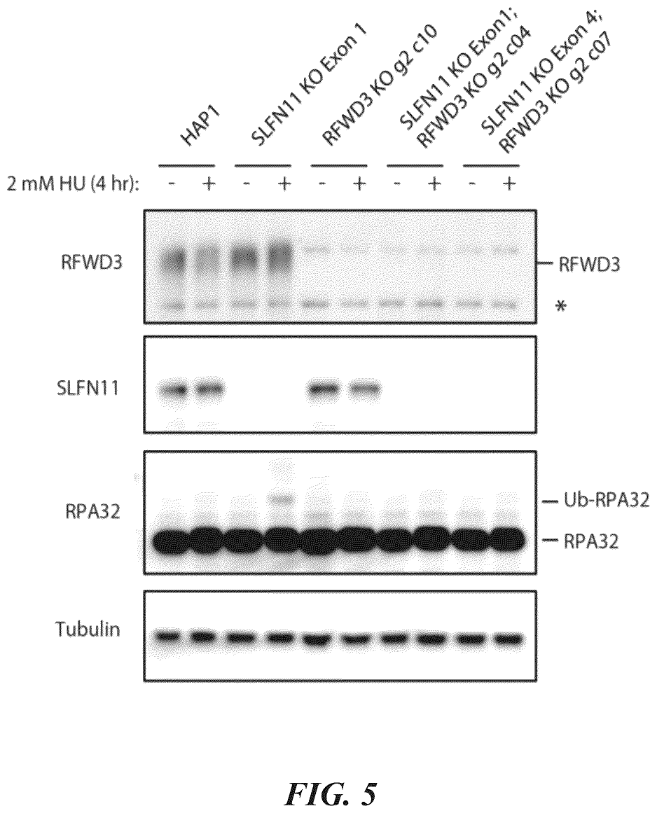

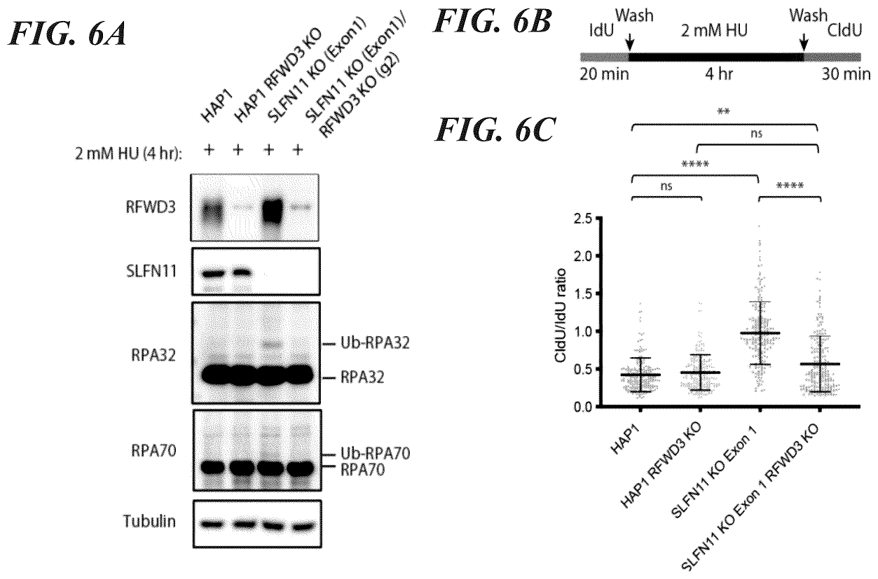

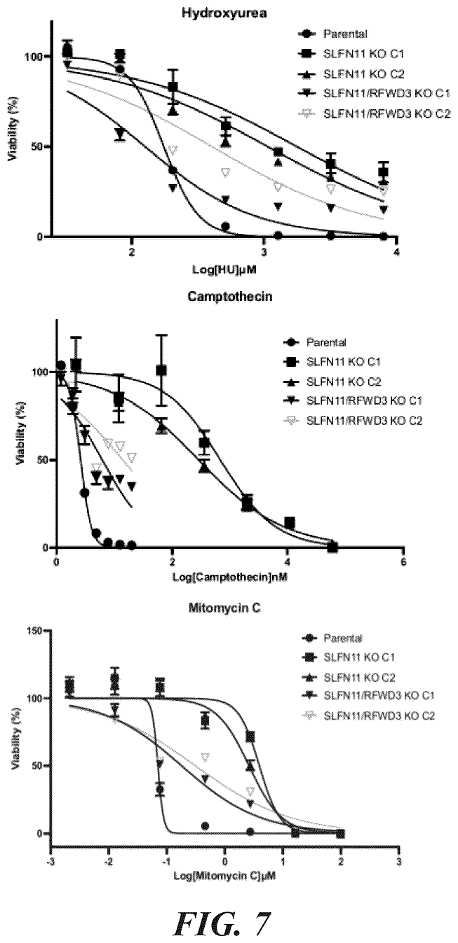

It was interrogated whether changes in replication stress signaling might be responsible for accelerated fork progression since it has previously been reported that SLFN11 suppresses signaling downstream of ATR. To test whether this was the case in HAP1 cells, parental HAP1 or SLFN11 KO cells were treated with 2 mM HU for 4 hours to induce ATR activation and then phosphosites were probed of canonical ATR substrates CHK1 (Ser345), RPA32 (Ser33 and Ser4/8) (FIG. 4). While no significant changes were observed in ATR substrate phosphorylation, a striking increase was observed in ubiquitinated RPA32 (Ub-RPA32). The E3 ubiquitin ligase responsible for RPA32 ubiquitination was previously identified as RFWD3. Knocking out RFWD3 in SLFN11 knockout cells to generate SLFN11/RFWD3 double knockout cell lines abrogated Ub-RPA32, consistent with prior reports in the literature implicating RPA32 as a substrate for RFWD3 (FIG. 5). SLFN11/RFWD3 double knockout cells (FIG. 6A) phenocopy the SLFN11 parental fork restart phenotype, suggesting that SLFN11 antagonizes RFWD3 activity (FIGS. 6A and 6C). SLFN11/RFWD3 double knockout cells exhibit sensitivity to camptothecin, mitomycin C, and hydroxyurea that is comparable to the parental, SLFN11 expressing cell lines (FIG. 7). This evidence suggests that targeting RFWD3 in the setting of SLFN11 low cancers can sensitize them to chemotherapy.

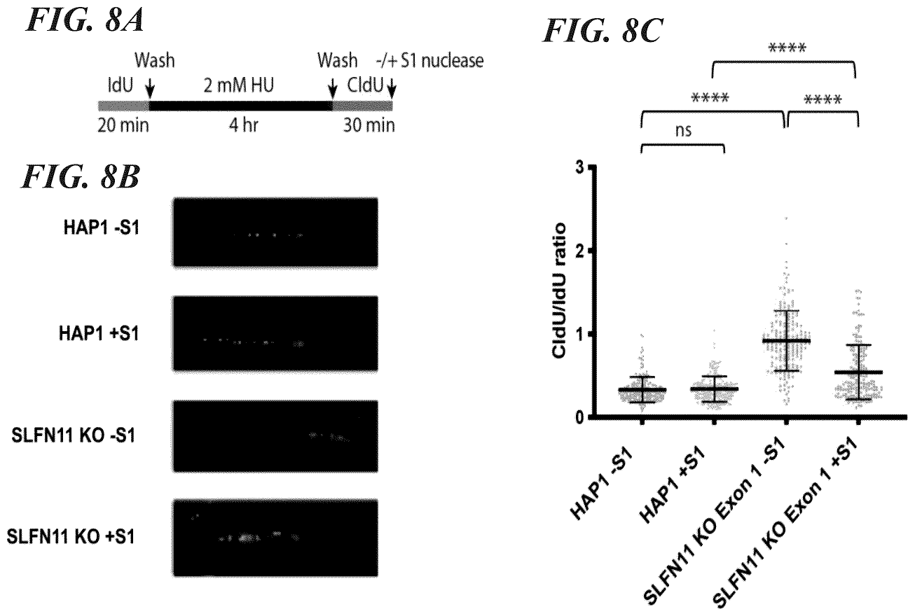

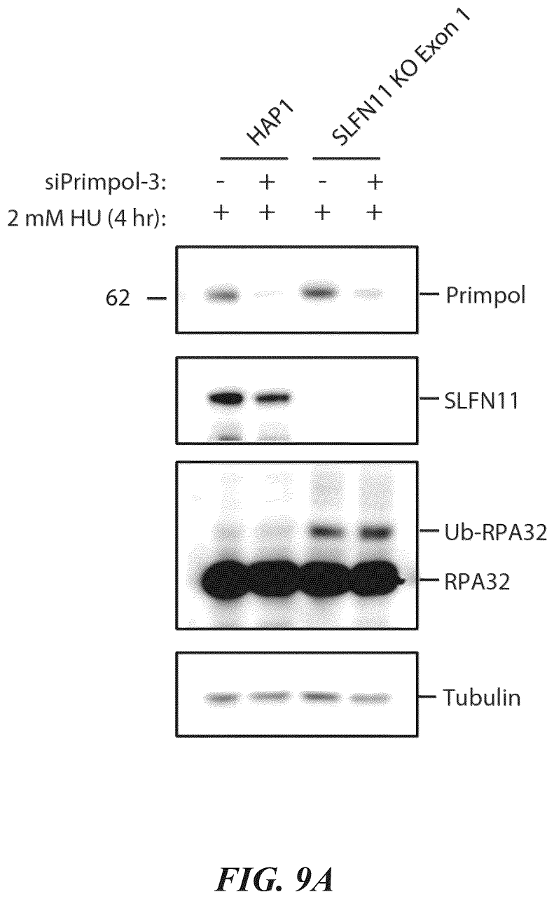

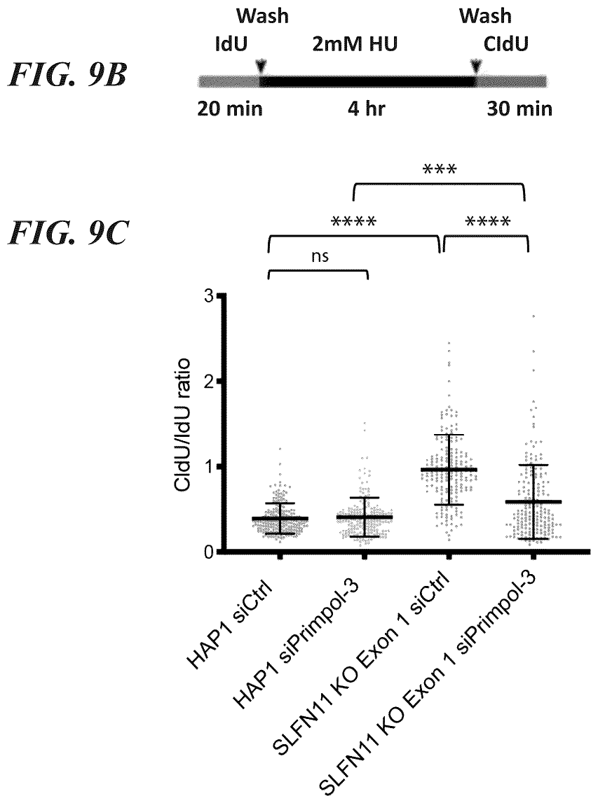

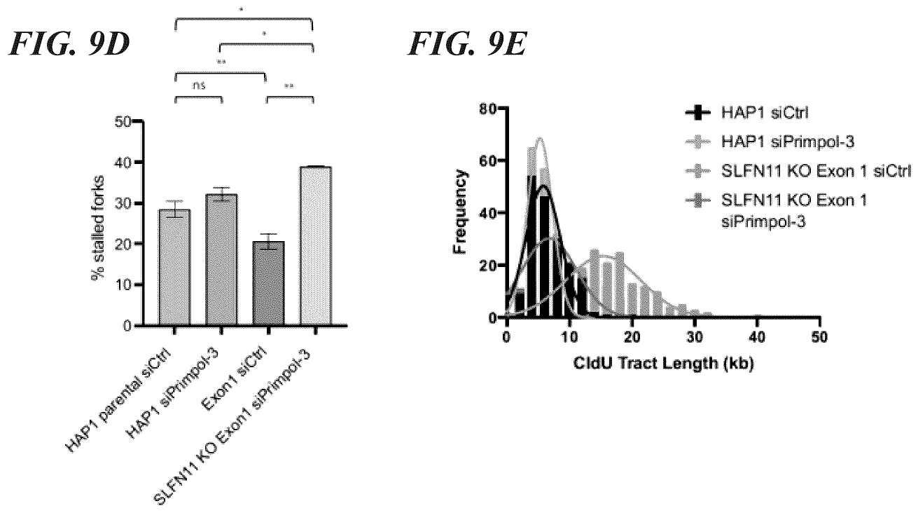

While RFWD3 has a previously described role in fork restart, it has not been previously appreciated how disrupting RFWD3 function in SLFN11-low cancer cell lines can promote chemosensitivity. It was hypothesized that the RFWD3-dependent accelerated fork progression in SLFN11 knockout cells might be due to unrestrained activity of PrimPol. To test this hypothesis, digested DNA fibers were digested with S1 nuclease, which can reveal ssDNA replication gaps by collapsing what appear to be extended CIdU replication tracks (FIG. 8A). S1 nuclease digestion revealed that SLFN11 CIdU tracks are replete with ssDNA gaps, implicating PrimPol (FIGS. 8B-C). The role of PrimPol was tested in unrestrained fork restart in SLFN11 knockout cells by depleting PrimPol with RNA interference. Even partial depletion of PrimPol in SLFN11 knockout cell lines phenocopied the parental, SLFN11-expressing cell lines as well as SLFN11/RFWD3 double knockout cell lines with respect to RPA32 ubiquitination (FIG. 9A), fork restart relative to initial fork speed (FIG. 9C), percent of stalled forks (FIG. 9D) and absolute tract length in kilobases (FIG. 9E).

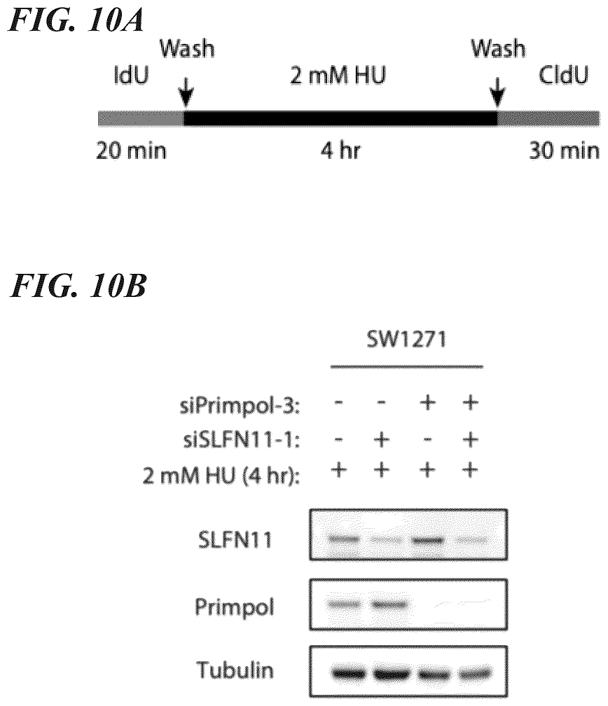

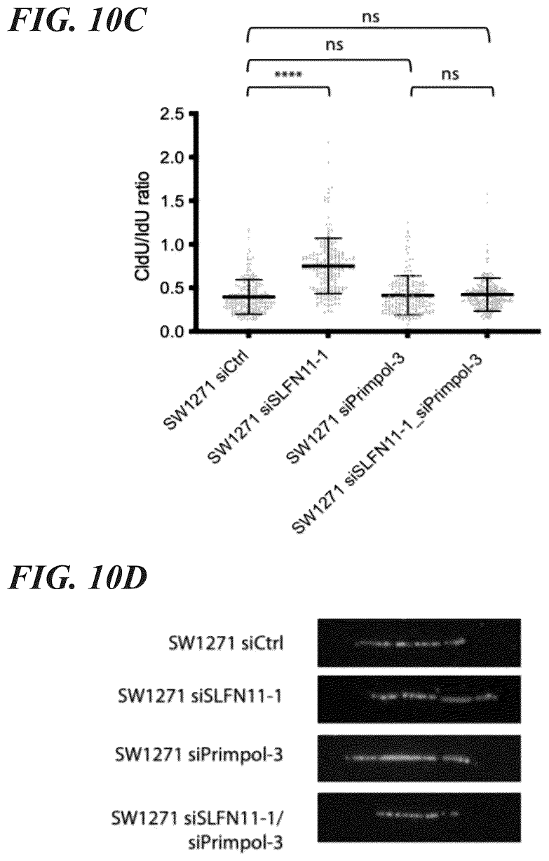

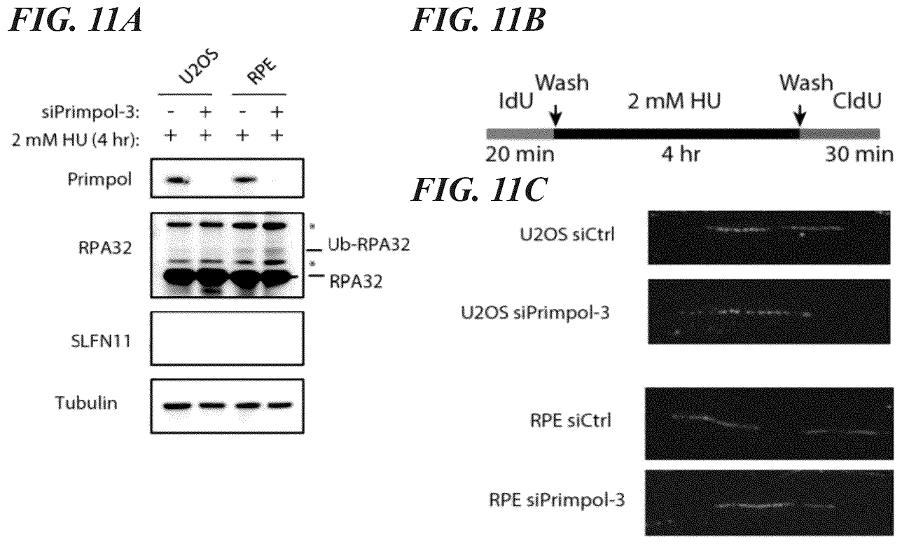

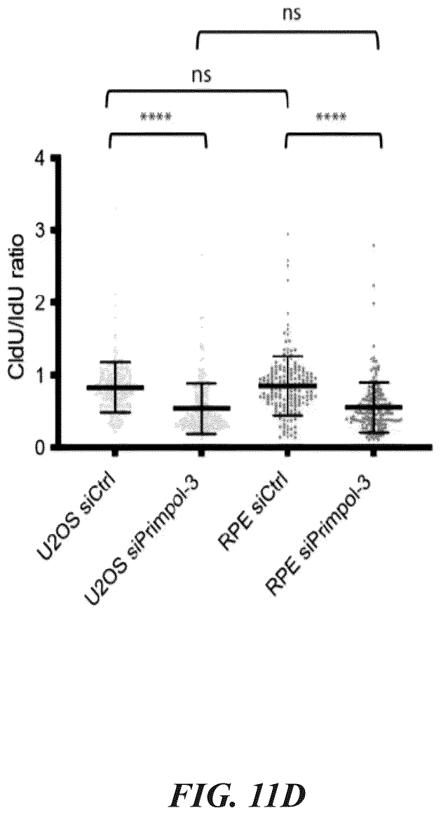

To test whether this mechanism was generalizable, SLFN11 and PrimPol RNAi experiments were performed in the small cell lung cancer cell line SW1271 and comparable results were observed, suggesting that this biology is not an artifact of a single cell line (FIG. 10). To test whether cell lines not known to have ever expressed SLFN11 rely on PrimPol for replication fork restart in a similar manner, we again used siRNA to knock down PrimPol in two cell lines that do not express SLFN11: U2OS and RPE cells (FIG. 11A). These results phenocopied HAP1 parental cell lines, which express SLFN11, and recapitulated the results observed when PrimPol or RFWD3 are disrupted in cells that have been manipulated to lack SLFN11 (FIGS. 11B-D).

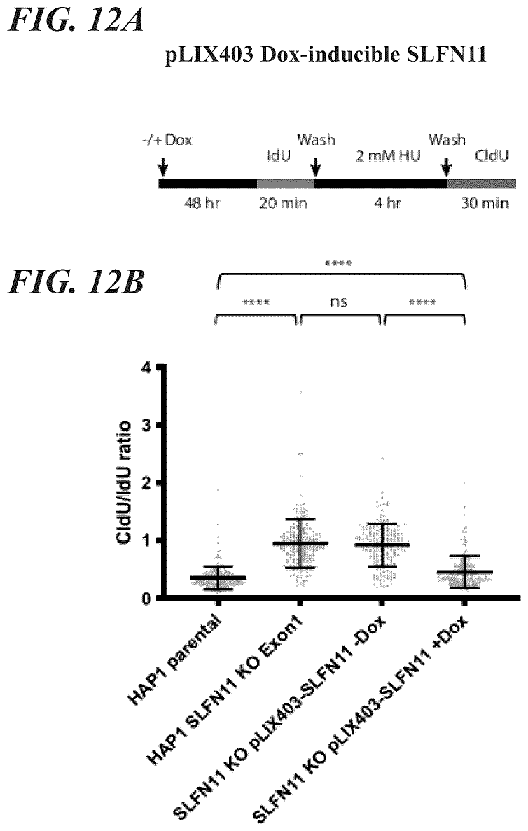

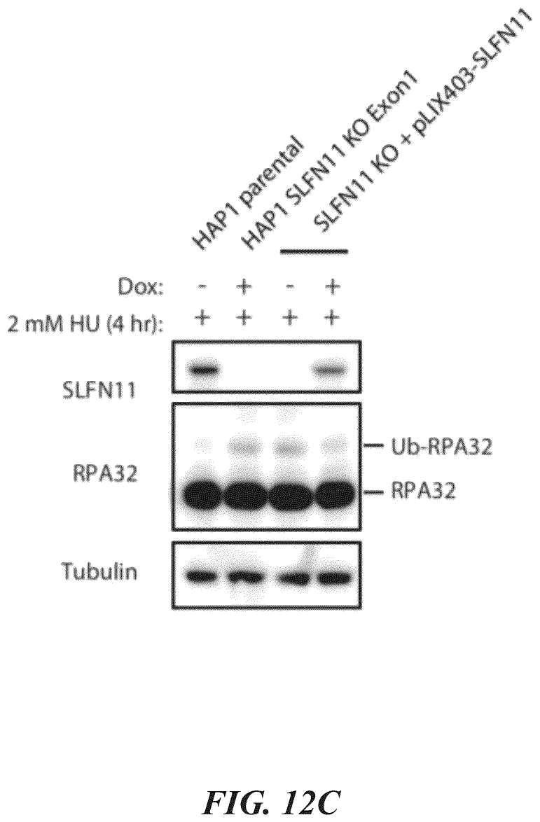

HAP1 knockout cells were transduced with lentivirus encoding full-length SLFN11 cDNA under the control of a doxycycline-inducible promoter and selected for stable integration of the construct using puromycin. In the presence of doxycycline, SLFN11 genetic knockout cells reconstituted with pLIX403-SLFN11 have the same fork restart defect as observed in the parental cell line (FIGS. 12A-B) express SLFN11 protein at comparable levels to the parental HAP1 cells, and show reduced levels of ubiquitinated RPA32 (FIG. 12C). This experiment confirms the sufficiency of SLFN11 rescue to antagonize RFWD3 in SLFN11 knockout cell lines.

Taken together, these results strongly suggest that chemosensitivity to many drugs is governed by SLFN11, which antagonizes the function of RFWD3 and PrimPol-both of which are required for unrestrained fork progression following stressed replication fork restart. Targeting RFWD3 and PrimPol pharmacologically is an attractive therapeutic strategy in SLFN11-low cancers, cancers with heterogeneous SLFN11 expression, or to suppress the emergence of SLFN11 low subclones.

### Example 2: Depletion of RFWD3 and PrimPol

Doxycycline-inducible shRNAs targeting either RFWD3 or PrimPol in multiple different cancer cell lines including cell lines that express SLFN11 natively, those that do not express SLFN11 or that have lost SLFN11 expression as a result of drug treatment, and isogenic SLFN11 knockout cell lines are conducted. It is hypothesized that depletion of these targets in vitro and in vivo will have the effect of increasing the depth and prolonging the duration of response to multiple chemotherapeutic agents that induce replication stress including topoisomerase poisons, PARP inhibitors, cisplatin, hydroxyurea and others.

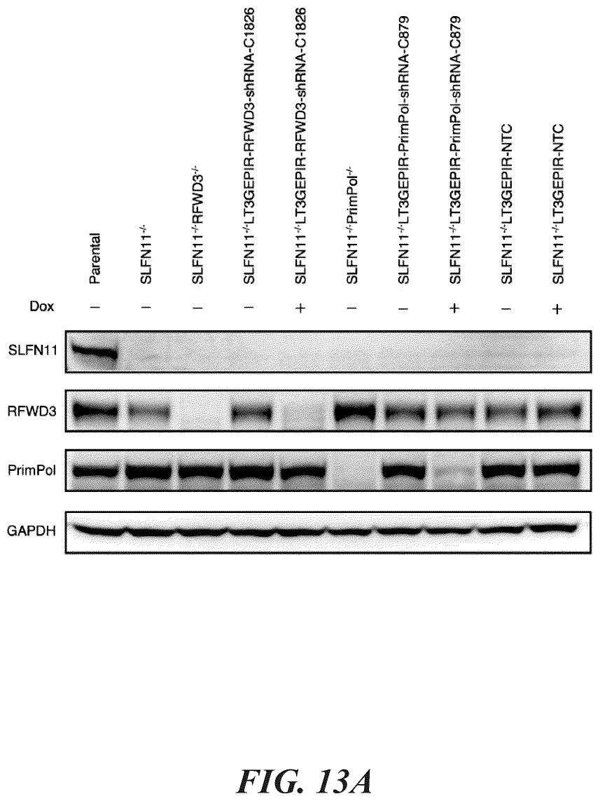

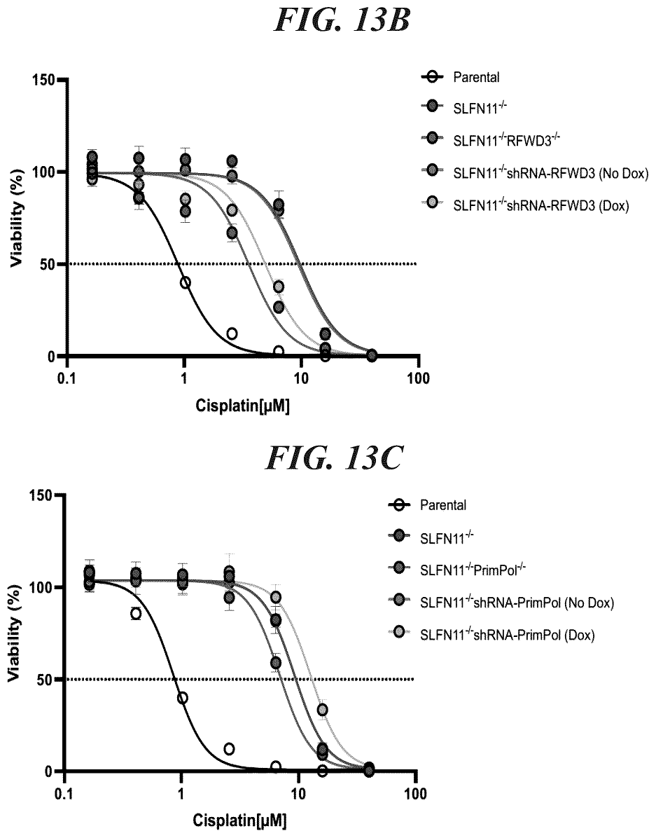

To test this hypothesis, 7-point dose-response curves to each of these agents were obtained in vitro in the presence or absence of doxycycline. The addition of doxycycline, which depleted either RFWD3 or PrimPol by RNA interference (FIG. 13A), was sufficient to sensitize cells that do not express SLFN11 to cisplatin (FIGS. 13B-C).

Similarly, tumor xenografts derived from these cell lines are established in immunocompromised mice. During tumorigenesis, mice are maintained on normal diet or chow containing doxycycline to induced RFWD3 or PrimPol depletion. When the tumors reach a volume of ˜150 mm³, a sub-therapeutic dose of one of the previously stated chemotherapeutic agents is administered. It is hypothesized that significantly higher therapeutic effect in the treatment arms in which animals are fed doxycycline chow and either RFWD3 or PrimPol is depleted. After the tumor growth curves diverge, chow treatment is swapped. It is then observed whether the treatment effect reverses between the two groups (i.e., tumors that saw no therapeutic effect begin to regress once placed on dox chow and vice versa).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for treating cancer in a subject, the method comprising:
(a) decreasing one or both of (i) an amount of an endogenous ubiquitin ligase in cancerous cells of a subject and (ii) ligase activity of the endogenous ubiquitin ligase in cancerous cells of the subject, wherein the endogenous ubiquitin ligase has at least 80% sequence identity to SEQ ID NO: 1; and
(b) administering a therapeutically effective amount of a chemotherapeutic agent to the subject.

2. The method of claim 1, wherein the subject has, prior to (a), been identified as having reduced expression or activity of Schlafen 11.

3. The method of claim 2, wherein reduced expression or activity of Schlafen 11 is determined by comparing a level of Schlafen 11 expression to a threshold value.

4. The method of any preceding claim, wherein the cancerous cells exhibit less than 1% Schlafen 11 positivity by immunochemistry.

5. The method of claim 2, wherein reduced expression or activity of Schlafen 11 is determined by comparing a level of Schlafen 11 activity to a threshold value.

6. The method of any preceding claim, wherein the method comprises decreasing an amount of the endogenous ubiquitin ligase in cancerous cells of the subject.

7. The method of claim 6, wherein decreasing the amount of the endogenous ubiquitin ligase comprises knocking out a gene that encodes for the endogenous ubiquitin ligase.

8. The method of claim 7, wherein the gene is knocked out via CRISPR, TALEN, or zinc-finger nuclease DNA editing.

9. The method of claim 6, wherein decreasing the amount of the endogenous ubiquitin ligase comprises post-transcriptional gene silencing (e.g., RNAi).

10. The method of claim 6, wherein decreasing the amount of the endogenous ubiquitin ligase comprises delivering a microRNA (miRNA) or a precursor thereof to the subject, wherein the miRNA is configured to bind to an mRNA encoding the endogenous ubiquitin ligase.

11. The method of claim 6, wherein decreasing the amount of the endogenous ubiquitin ligase comprises delivering an antisense oligonucleotide or a precursor thereof to the subject, wherein the antisense oligonucleotide is configured to bind to an mRNA encoding the endogenous ubiquitin ligase.

12. The method of any preceding claim, wherein the method comprises decreasing ligase activity of the endogenous ubiquitin ligase in cancerous cells of the subject.

13. The method of any preceding claim, wherein decreasing the activity of the endogenous ubiquitin ligase comprises administering an inhibitor of the endogenous ubiquitin ligase to the subject.

14. The method of any preceding claim, wherein the chemotherapeutic agent is a drug that induces replication stress during S phase.

15. The method of any preceding claim, wherein the chemotherapeutic agent is selected from the group consisting of a PARP inhibitor, a DNA alkylating agent, a DNA crosslinker, a topoisomerase I inhibitor, a topoisomerase II inhibitor, a polymerase inhibitor, a polymerase alpha inhibitor, and a clastogen.

16. The method of any preceding claim, wherein the cancerous cells exhibit unrestrained fork restart.

17. The method of any preceding claim, wherein the cancerous cells exhibit single-strand DNA replication gaps after fork restart.

18. The method of any preceding claim, wherein the cancerous cells are small cell lung cancer cells.

19. The method of any one of claims 1-18, wherein the cancerous cells are from a neuroendocrine tumor.

20. The method of claim 19, wherein the neuroendocrine tumor is a high-grade neuroendocrine tumor.

21. The method of any preceding claim, wherein the subject had been previously treated with a chemotherapeutic agent prior to (a), and the cancerous cells exhibited chemoresistance prior to (a).

22. The method of any preceding claim, wherein the endogenous ubiquitin ligase has at least 85%, at least 90%, at least 95%, at least 96%, at least 97%, at least 98%, at least 99%, or 100% sequence identity to SEQ ID NO: 1.

23. A method for sensitizing a plurality of cancerous cells to a chemotherapeutic agent, the method comprising:
(a) decreasing one of both of (i) an amount of an endogenous ubiquitin ligase in a plurality of cancerous cells or (ii) ligase activity of the endogenous ubiquitin ligase in the cancerous cells, wherein the endogenous ubiquitin ligase has at least 80% sequence identity to SEQ ID NO: 1; and
(b) contacting the cells with a chemotherapeutic agent.

24. The method of claim 23, wherein the cancerous cells have, prior to (a), been identified as having reduced expression or activity of Schlafen 11.

25. The method of claim 24, wherein reduced expression or activity of Schlafen 11 is determined by comparing a level of Schlafen 11 expression to a threshold value.

26. The method of any one of claims 23-25, wherein the cancerous cells exhibit less than 1% Schlafen 11 positivity by immunochemistry.

27. The method of claim 24, wherein reduced expression or activity of Schlafen 11 is determined by comparing a level of Schlafen 11 activity to a threshold value.

28. The method of any one of claims 23-27, wherein the method comprises decreasing an amount of the endogenous ubiquitin ligase in the cancerous cells.

29. The method of claim 28, wherein decreasing the amount of the endogenous ubiquitin ligase comprises knocking out a gene that encodes for the endogenous ubiquitin ligase.

30. The method of claim 29, wherein the gene is knocked out via CRISPR, TALEN, or zinc-finger nuclease DNA editing.

31. The method of claim 28, wherein decreasing the amount of the endogenous ubiquitin ligase comprises post-transcriptional gene silencing (e.g., RNAi).

32. The method of claim 28, wherein decreasing the amount of the endogenous ubiquitin ligase comprises delivering a microRNA (miRNA) or a precursor thereof to the cancerous cells, wherein the miRNA is configured to bind to an mRNA encoding the endogenous ubiquitin ligase.

33. The method of claim 28, wherein decreasing the amount of the endogenous ubiquitin ligase comprises delivering an antisense oligonucleotide or a precursor thereof to the cancerous cells, wherein the antisense oligonucleotide is configured to bind to an mRNA encoding the endogenous ubiquitin ligase.

34. The method of any one of claims 23-33, wherein the method comprises decreasing ligase activity of the endogenous ubiquitin ligase in the cancerous cells.

35. The method of any one of claims 23-34, wherein decreasing the activity of the endogenous ubiquitin ligase comprises administering an inhibitor of the endogenous ubiquitin ligase to the cancerous cells.

36. The method of any one of claims 23-35, wherein the chemotherapeutic agent is a drug that induces replication stress during S phase.

37. The method of any one of claims 23-36, wherein the chemotherapeutic agent is selected from the group consisting of: a PARP inhibitor, a DNA alkylating agent, a DNA crosslinker, a topoisomerase I inhibitor, a topoisomerase II inhibitor, a polymerase inhibitor, a polymerase alpha inhibitor, and a clastogen.

38. The method of any one of claims 23-37, wherein the cancerous cells exhibit unrestrained fork restart.

39. The method of any one of claims 23-38, wherein the cancerous cells exhibit single-strand DNA replication gaps after fork restart.

40. The method of any one of claims 23-39, wherein the cancerous cells are small cell lung cancer cells.

41. The method of any one of claims 23-39, wherein the cancerous cells are from a neuroendocrine tumor.

42. The method of claim 41, wherein the neuroendocrine tumor is a high-grade neuroendocrine tumor.

43. The method of any one of claims 23-42, wherein the cancerous cells had been previously treated with a chemotherapeutic agent prior to (a), and the cancerous cells exhibited chemoresistance prior to (a).

44. The method of 23-43, wherein the endogenous ubiquitin ligase has at least 85%, at least 90%, at least 95%, at least 96%, at least 97%, at least 98%, at least 99%, or 100% sequence identity to SEQ ID NO: 1.

45. A method for treating cancer in a subject, the method comprising:
(a) decreasing one or both of (i) an amount of an endogenous enzyme having DNA polymerase and DNA primase activity (PrimPol) in cancerous cells of a subject and (ii) primase and/or polymerase activity of the endogenous enzyme having DNA polymerase and DNA primase activity in cancerous cells of the subject, wherein the enzyme has at least 80% sequence identity to SEQ ID NO: 2; and
(b) administering a therapeutically effective amount of a chemotherapeutic agent to the subject.

46. The method of claim 45, wherein the subject has, prior to (a), been identified as having reduced expression or activity of Schlafen 11.

47. The method of claim 46, wherein reduced expression or activity of Schlafen 11 is determined by comparing a level of Schlafen 11 expression to a threshold value.

48. The method of any one of claims 45-47, wherein the cancerous cells exhibit less than 1% Schlafen 11 positivity by immunochemistry.

49. The method of claim 46, wherein reduced expression or activity of Schlafen 11 is determined by comparing a level of Schlafen 11 activity to a threshold value.

50. The method of any one of claims 45-49, wherein the method comprises decreasing an amount of the enzyme in cancerous cells of the subject.

51. The method of claim 50, wherein decreasing the amount of the enzyme comprises knocking out a gene that encodes for the enzyme.

52. The method of claim 51, wherein the gene is knocked out via CRISPR, TALEN, or zinc-finger nuclease DNA editing.

53. The method of claim 50, wherein decreasing the amount of the enzyme comprises post-transcriptional gene silencing (e.g., RNAi).

54. The method of claim 50, wherein decreasing the amount of the enzyme comprises delivering a microRNA (miRNA) or a precursor thereof to the subject, wherein the miRNA is configured to bind to an mRNA encoding the enzyme.

55. The method of claim 50, wherein decreasing the amount of the enzyme comprises delivering an antisense oligonucleotide or a precursor thereof to the subject, wherein the antisense oligonucleotide is configured to bind to an mRNA encoding the enzyme.

56. The method of any one of claims 45-55, wherein the method comprises decreasing primase activity of an enzyme in cancerous cells of the subject.

57. The method of any one of claims 45-56, wherein the method comprises decreasing polymerase activity of an enzyme in cancerous cells of the subject.

58. The method of any one of claims 45-57, wherein decreasing the activity of the enzyme comprises administering an inhibitor of the enzyme to the subject.

59. The method of any one of claims 45-58, wherein the chemotherapeutic agent is a drug that induces replication stress during S phase.

60. The method of any one of claims 45-59, wherein the chemotherapeutic agent is selected from the group consisting of: a PARP inhibitor, a DNA alkylating agent, a DNA crosslinker, a topoisomerase I inhibitor, a topoisomerase II inhibitor, a polymerase inhibitor, a polymerase alpha inhibitor, and a clastogen.

61. The method of any one of claims 45-60, wherein the cancerous cells exhibit unrestrained fork restart.

62. The method of any one of claims 45-61, wherein the cancerous cells exhibit single-strand DNA replication gaps after fork restart.

63. The method of any one of claims 45-62, wherein the cancerous cells are small cell lung cancer cells.

64. The method of any one of claims 45-62, wherein the cancerous cells are from a neuroendocrine tumor.

65. The method of claim 64, wherein the neuroendocrine tumor is a high-grade neuroendocrine tumor.

66. The method of any one of claims 45-65, wherein the subject had been previously treated with a chemotherapeutic agent prior to (a), and the cancerous cells exhibited chemoresistance prior to (a).

67. The method of any one of claims 45-66, wherein the enzyme has at least 85%, at least 90%, at least 95%, at least 96%, at least 97%, at least 98%, at least 99%, or 100% sequence identity to SEQ ID NO: 2.

68. A method for sensitizing a plurality of cancerous cells to a chemotherapeutic agent, the method comprising:
(a) decreasing one of both of (i) an amount of an endogenous enzyme having DNA polymerase and DNA primase activity (PrimPol) in a plurality of cancerous cells or (ii) primase and/or polymerase activity of the endogenous enzyme having DNA polymerase and/or DNA primase activity in the cancerous cells, wherein the enzyme has at least 80% sequence identity to SEQ ID NO: 2; and
(b) contacting the cells with a chemotherapeutic agent.

69. The method of claim 68, wherein the cancerous cells have, prior to (a), been identified as having reduced expression or activity of Schlafen 11.

70. The method of claim 69, wherein reduced expression or activity of Schlafen 11 is determined by comparing a level of Schlafen 11 expression to a threshold value.

71. The method of any one of claims 68-70, wherein the cancerous cells exhibit less than 1% Schlafen 11 positivity by immunochemistry.

72. The method of claim 69, wherein reduced expression or activity of Schlafen 11 is determined by comparing a level of Schlafen 11 activity to a threshold value.

73. The method of any one of claims 68-72, wherein the method comprises decreasing an amount of an enzyme in the cancerous cells.

74. The method of claim 73, wherein decreasing the amount of the enzyme knocking out a gene that encodes for the enzyme.

75. The method of claim 74, wherein the gene is knocked out via CRISPR, TALEN, or zinc-finger nuclease DNA editing.

76. The method of claim 73, wherein decreasing the amount of the enzyme comprises post-transcriptional gene silencing (e.g., RNAi).

77. The method of claim 73, wherein decreasing the amount of the enzyme comprises delivering a microRNA (miRNA) or a precursor thereof to the cancerous cells, wherein the miRNA is configured to bind to an mRNA encoding the enzyme.

78. The method of claim 73, wherein decreasing the amount of the enzyme comprises delivering an antisense oligonucleotide or a precursor thereof to the cancerous cells, wherein the antisense oligonucleotide is configured to bind to an mRNA encoding the enzyme.

79. The method of any one of claims 68-78, wherein the method comprises decreasing DNA primase activity of an enzyme in the cancerous cells.

80. The method of any one of claims 68-79, wherein the method comprises decreasing DNA polymerase activity of an enzyme in the cancerous cells.

81. The method of any one of claims 68-80, wherein decreasing the activity of the enzyme comprises administering an inhibitor of the enzyme to the cancerous cells.

82. The method of any one of claims 68-81, wherein the chemotherapeutic agent is a drug that induces replication stress during S phase.

83. The method of any one of claims 68-82, wherein the chemotherapeutic agent is selected from the group consisting of: a PARP inhibitor, a DNA alkylating agent, a DNA crosslinker, a topoisomerase I inhibitor, a topoisomerase II inhibitor, a polymerase inhibitor, a polymerase alpha inhibitor, and a clastogen.

84. The method of any one of claims 68-83, wherein the cancerous cells exhibit unrestrained fork restart.

85. The method of any one of claims 68-84, wherein the cancerous cells exhibit single-strand DNA replication gaps after fork restart.

86. The method of any one of claims 68-85, wherein the cancerous cells are small cell lung cancer cells.

87. The method of any one of claims 68-86, wherein the cancerous cells are from a neuroendocrine tumor.

88. The method of claim 87, wherein the neuroendocrine tumor is a high-grade neuroendocrine tumor.

89. The method of any one of claims 68-88, wherein the cancerous cells had been previously treated with a chemotherapeutic agent prior to (a), and the cancerous cells exhibited chemoresistance prior to (a).

90. The method of any one of claims 68-89, wherein the enzyme has at least 85%, at least 90%, at least 95%, at least 96%, at least 97%, at least 98%, at least 99%, or 100% sequence identity to SEQ ID NO: 2.
